(19) United States
(12) Patent Application Publication
(10) Pub. No.: US 2020/0011847 A1
(43) Pub. Date: Jan. 9, 2020

(54) ANALYSIS SYSTEM AND METHOD FOR TESTING A SAMPLE

(71) Applicant: BOEHRINGER INGELHEIM VETMEDICA GMBH, Ingelheim am Rhein (DE)

(72) Inventors: Guenter BRUCKMANN, Wuerselen (DE); Erol MEYDA, Aachen (DE); Axel NIEMEYER, Bielefeld (DE); Harald PAULS, Eschweiler (DE); Hannah SCHMOLKE, Didderse (DE)

(73) Assignee: BOEHRINGER INGELHEIM VETMEDICA GMBH, Ingelheim am Rhein (DE)

(21) Appl. No.: 16/337,424

(22) PCT Filed: Oct. 5, 2017
(86) PCT No.: PCT/EP2017/025290

§ 371 Date: Mar. 28, 2019

(30) Foreign Application Priority Data
Oct. 7, 2016 (EP) 16020382.4

(51) Int. Cl. G01N 33/487; G01N 27/22; G01N 35/00

(52) U.S. Cl. CPC G01N 33/487; G01N 35/021; G01N 35/00693; G01N 27/22

(57) ABSTRACT

An analysis system and a method for testing a biological sample is provided, wherein the sensitivity of the evaluation electronics of a fluid sensor is specified and/or changed depending on a phase of the test sequence and/or a cartridge identifier of the cartridge, and/or in that the fluid sensor comprises a sensor electrode that is intended for measuring electrical capacitance and is operated in a manner in which it is electrically connected to the evaluation electronics by a single pole and/or by means of a shielded sensor line.

### BACKGROUND OF THE INVENTION

### Field of the Invention

The present invention relates to methods for testing a sample using an analysis system which includes a cartridge and an analysis device having a fluid sensor with evaluation electronics, and to a computer program product for executing the methods, and to analysis systems for testing a sample using an analysis system which includes a cartridge and an analysis device having a fluid sensor with evaluation electronics.

Preferably, the present invention deals with analyzing and testing a sample, in particular from a human or animal, particularly preferably for analytics and diagnostics, for example with regard to the presence of diseases and/or pathogens and/or for determining blood counts, antibodies, hormones, steroids or the like. Therefore, the present invention is in particular within the field of bioanalytics. A food sample, environmental sample or another sample may optionally also be tested, in particular for environmental analytics or food safety and/or for detecting other substances.

Preferably, by means of the present invention, at least one analyte (target analyte) of a sample can be determined, identified or detected. In particular, the sample can be tested for qualitatively or quantitatively determining at least one analyte, for example in order for it to be possible to detect or identify a disease and/or pathogen.

Within the meaning of the present invention, analytes are in particular nucleic-acid sequences, in particular DNA sequences and/or RNA sequences, and/or proteins, in particular antigens and/or antibodies. In particular, by means of the present invention, nucleic-acid sequences can be determined, identified or detected as analytes of a sample, and/or proteins can be determined, identified or detected as analytes of the sample. More particularly preferably, the present invention deals with systems, devices and other apparatuses for carrying out a nucleic-acid assay for detecting or identifying a nucleic-acid sequence and/or a protein assay for detecting or identifying a protein.

The present invention deals in particular with what are known as point-of-care systems, i.e. in particular with mobile systems, devices and other apparatuses, and deals with methods for carrying out tests on a sample at the sampling site and/or independently and/or away from a central laboratory or the like. Preferably, point-of-care systems can be operated autonomously of and/or independently from a mains network for supplying electrical power.

### Description of the Related Art

U.S. Pat. No. 5,096,669 discloses a point-of-care system for testing a biological sample, in particular a blood sample. The system comprises a single-use cartridge and an analysis device. Once the sample has been received, the cartridge is inserted into the analysis device in order to carry out the test. The cartridge comprises a microfluidic system and a sensor apparatus comprising electrodes, which apparatus is calibrated by means of a calibration liquid and is then used to test the sample.

Furthermore, International Publication No. WO 2006/125767 A1 and corresponding US Patent Application Publication No. 2009/0298059 A1 disclose a point-of-care system for integrated and automated DNA or protein analysis, comprising a single-use cartridge and an analysis device for fully automatically processing and evaluating molecular-diagnostic analyses using the single-use cartridge. The cartridge is designed to receive a sample, in particular blood, and in particular allows cell disruption, PCR and detection of PCR amplification products, which are bonded to capture molecules and provided with a label enzyme, in order for it to be possible to detect bonded PCR amplification products or nucleic sequences as target analytes in what is known as a redox cycling process. A capacitative level sensor is disclosed having two plates. However, there is no hint regarding how to achieve a more reliable detection or less complex construction.

German Patent No. DE 100 58 394 C1 and corresponding U.S. Pat. No. 7,838,261 B2 disclose a method for testing a sample using a reaction array comprising at least two reaction compartments for receiving substances that react with one another, the reaction compartments being interconnected by means of a supply space. In order to measure the substances, an exchange of substances and thus chemical crosstalk between the individual reaction compartments is prevented by lowering a sensor cover. In this way, the detection sensitivity of the method is increased.

European Patent No. EP 2 305 383 B1 and corresponding US Patent Application Publication No. 2008/0207461 A1 disclose an instrument for carrying out and analyzing microarray experiments. In particular, this document discloses carrying out microarray experiments in parallel in order to detect specific interactions between probe molecules and target molecules in a microtiter plate. In this case, probes in the form of a substance library are provided on carriers, and therefore a sample can be simultaneously analyzed on a plurality of probes in parallel. In the context of microarray experiments of this kind, it is also disclosed that a desired operating mode can be specified for a processing apparatus externally, in particular by a user. However, there is no hint regarding any sensor portion through which a sample can flow, and no hint how to improve detecting a content change within such sensor portion.

“Multielectrode capacitors” In Larry K. Baxter: “Capacitive Sensors: Design and Applications”, 31 Aug. 1996, Wiley-IEEE Press discloses in chapter 2.3.2 “Multielectrode capacitors” an air spaced capacitor having three electrodes, one of which is connected to ground for shielding. The chapter relates to multielectrode capacitors, i.e. having more than two nodes, and there is no hint regarding connecting capacitor electrodes. Moreover, the capacitor electrodes are shielded in their entirely and, thus, are not suitable for sensing purposes.

### SUMMARY OF THE INVENTION

The problem addressed by the present invention is to provide a method, an analysis system and a computer program product for more accurately or reliably testing a sample.

The above problem is solved by a method for testing a sample by means of an analysis system, including receiving the sample in a cartridge of the analysis system, the cartridge comprising a fluid system that has a sensor portion through which fluid can flow, and receiving the cartridge by an analysis device of the analysis system, and subsequently carrying out a test using the received cartridge, the analysis device including at least one fluid sensor that has evaluation electronics for detecting a content change in the sensor portion, wherein the sensitivity of the evaluation electronics is at least one of specified or changed depending on one or more of a phase of a test sequence and a cartridge identifier of the cartridge. The above problem is also solved by a method for testing a sample by means of an analysis system, including receiving the sample in a cartridge of the analysis system, the cartridge comprising a fluid system that has a sensor portion through which fluid can flow, and receiving the cartridge by an analysis device of the analysis system, and subsequently carrying out a test using the received cartridge, the analysis device including at least one fluid sensor that has evaluation electronics for detecting a content change in the sensor portion, wherein the analysis system is configured to at least one of specify or change the sensitivity of the evaluation electronics depending on one or more of a phase of the test sequence and a cartridge identifier of the cartridge. The above problem is also solved by a computer program product for carrying out the above-noted methods. The above problem is also solved by an analysis system for testing a sample, a cartridge for receiving the sample, the cartridge comprising a fluid system that has a sensor portion through which fluid can flow, and an analysis device for receiving the cartridge and subsequently carrying out a test using the received cartridge, the analysis device comprising at least one fluid sensor that has evaluation electronics for detecting a content change in the sensor portion, wherein the analysis system is configured to at least one of specify or change the sensitivity of the evaluation electronics depending on one or more of a phase of the test sequence and a cartridge identifier of the cartridge The above problem is also solved by an analysis system for testing a sample, a cartridge for receiving the sample, the cartridge comprising a fluid system that has a sensor portion through which fluid can flow, and an analysis device for receiving the cartridge and subsequently carrying out a test using the received cartridge, the analysis device comprising at least one fluid sensor that has evaluation electronics for detecting a content change in the sensor portion, wherein the fluid sensor comprises a sensor electrode that is configured for measuring electrical capacitance and is at least one of single ended or electrically connected to the evaluation electronics by a shielded sensor line.

The present invention relates to the testing of an in particular biological sample using an analysis system. The analysis system preferably comprises a cartridge for receiving the sample. The cartridge preferably comprises a fluid system that has a sensor portion through which fluid can flow.

It is preferable for the analysis system to comprise an analysis device for receiving the cartridge and subsequently carrying out the test using the received cartridge.

The analysis device preferably comprises a fluid sensor that has evaluation electronics for detecting a content change in the sensor portion of the cartridge. In particular, the fluid sensor is designed to detect a fluid and/or a liquid front, in particular the sample, entering or leaving the sensor portion.

The fluid sensor preferably measures, preferably as a measurement result, an electrical variable, in particular capacitance, which is dependent on a property, in particular electrical permittivity and/or electrical conductivity and/or electrical permeability, of the content of the sensor portion. In other words, the fluid sensor can therefore preferably be influenced by the content of the sensor portion such that electrical properties can be changed and detected. This does not mean that the sensor portion content itself has to be conductive or electrically active in another manner, or that electrical current has to flow therethrough, even though this is possible in principle.

If the measurement result changes, it is preferably concluded that there has been a content change in the sensor portion. This conclusion can be drawn by the measurement result being compared with a reference value and the content change being detected if the reference value is exceeded. The actual value is therefore preferably compared with the desired value and/or with a threshold value.

In a first aspect of the present invention, the sensitivity of the evaluation electronics is specified and/or changed depending on a phase of the test sequence. Alternatively or additionally, it is provided that the sensitivity of the evaluation electronics is specified and/or changed depending on a cartridge identifier of the cartridge.

The sensitivity of the evaluation electronics is preferably fixed or influenced by a reference value/the reference value for comparison with the measurement result. Alternatively or additionally, the sensitivity of the evaluation electronics is determined or influenced by the gain by which a signal that can be influenced by the content of the sensor portion is amplified.

In particular, a sensor electrode is provided that is connected to a measuring amplifier, the electrical properties of the sensor electrode being dependent on the content of the sensor portion, and the gain of the measuring amplifier and/or a reference value for comparison with the measured value preferably being specified, provided, settable and/or set.

More particularly preferably, the sensor electrode is an electrode that is, for example, flat or planar at least on a side that faces the cartridge, in particular is a capacitor plate that is arranged adjacently to the sensor portion such that the permittivity of the content of the sensor portion influences the capacitance of the sensor electrode.

The reference value and/or the gain is/are preferably dependent on the phase of the test sequence. Alternatively or additionally, the reference value and/or the gain is/are dependent on the identifier of the cartridge. Particularly preferably, the reference value and/or the gain is/are specified and/or changed according to the phase of the test sequence and/or according to the identifier of the cartridge. Alternatively or additionally, the analysis system, the analysis device and/or the fluid sensor is designed for this purpose.

In one aspect of the present invention, calibration data and/or calibration information corresponding to the cartridge is/are stored, in particular as part of control information for controlling the test using the analysis device and the cartridge.

The cartridge preferably comprises an identifier which corresponds to the cartridge or to a batch of cartridges with which the cartridge is associated. Using this identifier, calibration information corresponding to the cartridge can be retrieved. Using the calibration information, it is possible to set the sensitivity of the evaluation electronics. The calibration information therefore preferably specifies the sensitivity, the reference value and/or the gain, in particular differently for different phases of the test.

Using the proposed method, a liquid front moving within the sensor portion is preferably detected. The test on the sample, in particular the conveyance of the sample and/or actuation of valves of the cartridge, is preferably controlled in a manner dependent on the detection of the liquid front and/or of a content change in the sensor portion.

Another aspect of the present invention, which can also be implemented independently, relates to a computer program product comprising program code means which, when executed, cause the method steps of the proposed method to be carried out. The computer program product can preferably be executed on a processor and/or controller of the analysis system, analysis device and/or operating instrument such that the proposed method can be or is carried out in its entirety or in part. The computer program product preferably is a non-transitory computer-readable media.

In one aspect, which can also be implemented independently, the present invention also preferably relates to an analysis system/the analysis system for carrying out the method. Here, the analysis system is preferably designed to specify and/or control the sensitivity of the evaluation electronics according to a phase of the test sequence and/or according to the identifier of the cartridge.

Another aspect of the present invention, which can also be implemented independently, relates to the construction of the fluid sensor and to a corresponding method for operating the fluid sensor. Here, it is preferably provided that the fluid sensor comprises the sensor electrode for measuring electrical capacitance, the sensor electrode being single ended (forming a single or being unipolar) and/or being connected to the evaluation electronics by means of a shielded sensor line.

The evaluation electronics is preferably designed to measure the electrical capacitance of the sensor electrode, in particular with respect to the surroundings of the sensor electrode, preferably without using a reference electrode or counter electrode.

The sensor electrode is therefore particularly preferably operated according to what is known as single-ended operation, for which no counter electrode is used. Instead, the fluid sensor preferably takes advantage of the fact that the ability of a conductive arrangement, in this case the sensor electrode, to store electrons is dependent on the dielectric properties of the medium surrounding said arrangement.

Therefore, in the preferred single-ended operation, the capacitance of the sensor electrode, i.e. the ability to absorb or store electrons, is dependent on the electrical and/or dielectrical properties of the content of the sensor portion. This relationship is utilized in that it is possible to determine the capacitance and to detect a content change therefrom when the capacitance changes.

As mentioned previously, the sensor electrode is preferably connected to the evaluation electronics by means of a sensor line. The sensor line is preferably provided adjacently to a shield electrode. The shield electrode is in particular connected to a fixed potential or (low level) earth. In this case, an at least substantially constant capacitance can be produced between the sensor line and the shield electrode.

By arranging the shield electrode adjacently to the sensor line, a sensor line capacitance and/or capacitor is preferably produced that is at least substantially independent from the surroundings of the sensor line. This makes it possible to prevent the capacitance measurement from being distorted or influenced by the influence or coupling in of disturbances, such as interference fields.

The evaluation electronics is preferably designed to measure the capacitance and/or compensate for the capacitance and/or capacitor produced between the sensor line and the shield electrode, and/or to take into account or ignore said capacitance and/or capacitor in the evaluation.

The evaluation electronics is in particular designed to carry out what is known as offset adjustment. In this manner, the constant electrical capacitance can be treated as so-called “offset” or can be compensated for in order to detect changes in capacitance starting from the constant capacitance. In this way, possible tolerances of the at least substantially constant capacitance or capacitance component of the arrangement, i.e. the sensor electrode and the sensor line, can be disregarded.

On the one hand, this prevents manufacturing tolerances from having an effect on the measurement and, on the other hand, the evaluation of the measurement results is not influenced by the at least substantially constant electrical capacitance or the offset. As a result, even when there are tolerances that have an effect on the constant capacitance proportion, it is possible to detect a change in capacitance and/or a content change in the sensor portion in a very accurate and/or reliable manner, for example by means of a reference value, in particular a threshold value, which only relates to the change in capacitance.

The sensor line connects the sensor electrode to the evaluation electronics preferably by means of a via in a printed circuit board. The analysis device therefore preferably comprises a printed circuit board, one side of which supports the sensor electrode and the other side of which supports the evaluation electronics, preferably each on one of the opposing flat sides of the printed circuit board.

In one aspect of the present invention, the sensor line is shielded in the region of the via. This can be achieved by a shield electrode/the shield electrode. Particularly preferably, the sensor line is guided in the via or in the region of the via, coaxially with the shield electrode. These measures make it possible to prevent or suppress both external disturbances and changes in capacitance that are not caused by a content change in the sensor portion.

The term “analysis device” is preferably understood to mean an instrument which is in particular mobile and/or can be used on site, and/or which is designed to chemically, biologically and/or physically test and/or analyze a sample or a component thereof, preferably in and/or by means of a cartridge. In particular, the analysis device controls the pretreatment and/or testing of the sample in the cartridge. For this purpose, the analysis device can act on the cartridge, in particular such that the sample is conveyed, temperature-controlled and/or measured in the cartridge.

The term “cartridge” is preferably understood to mean a structural apparatus or unit designed to receive, to store, to physically, chemically and/or biologically treat and/or prepare and/or to measure a sample, preferably in order to make it possible to detect or determine at least one analyte, in particular a protein and/or a nucleic-acid sequence, of the sample.

A cartridge within the meaning of the present invention preferably comprises a fluid system having a plurality of channels, cavities and/or valves for controlling the flow through the channels and/or cavities.

In particular, within the meaning of the present invention, a cartridge is designed to be at least substantially planar and/or card-like, in particular is designed as a (micro)fluidic card and/or is designed as a main body or container that can preferably be closed and/or said cartridge can be inserted and/or plugged into a proposed analysis device when it contains the sample.

The term “operating instrument” is preferably understood to mean an apparatus by means of which the analysis device can be controlled, control information can be transmitted to the analysis device, and/or measurement results can be received from the analysis device and/or measurement results can be evaluated. Preferably, the operating instrument is or forms a user interface for controlling the test and/or the evaluation or outputting of measurement results.

The operating instrument can alternatively be called operator control instrument. The operating instrument preferably is configured to be operated by an operator (user) for controlling, in particular of the analysis device, the test and/or the evaluation. Thus, the operating instrument is or comprises a user interface for input of commands and transfer of pieces of control information to the analysis device.

The operating instrument preferably comprises an input apparatus for controlling the analysis device, for controlling data transmission and/or for controlling the evaluation of measurement results. Alternatively or additionally, the operating instrument comprises an output apparatus for outputting, in particular displaying, information, in particular status information, operating elements and/or results. The operating instrument preferably comprises a processor, microcontroller and/or memory for executing a computer program product for data transmission, for control and/or for evaluating measurement results.

Particularly preferably, the operating instrument is a mobile terminal device, in particular for a radio and/or mobile network, such as a smartphone, tablet computer, mobile telephone or the like. The operating instrument can preferably be operated independently from a power network, using a power storage means, in particular a (rechargeable) battery, and in a mobile manner, autonomously of and/or independently from further components of the analysis system, in particular the analysis device. The operating instrument preferably comprises one or more interfaces for wireless data communications, in particular a WPAN communication interface, a WLAN communication interface, a near-field communication interface, an optical communication interface such as a camera, and/or a mobile radio interface.

The term “test” as used herein preferably means a test procedure, test sequence and/or performing an assay, in particular one, several or all steps for performing an assay to determine one or more analytes of a sample. The steps are preferably realized by or within the analysis system, analysis device and/or cartridge.

An “assay” according to the present invention is preferably an investigative procedure for qualitatively and/or quantitatively measuring, detecting and/or identifying the presence, amount, and/or functional activity of a target entity or analyte of the sample. The analyte can, e.g., be a drug, a biological, chemical and/or biochemical substance, and/or a cell in an organism or organic sample. In particular, the analyte can be a molecule, a nucleic-acid sequence, a DNA, an RNA and/or a protein.

Preferably, the assay according to the present invention is a nucleic-acid assay for detecting or identifying a nucleic-acid sequence and/or a protein assay for detecting or identifying a protein.

An assay, test or test procedure according to the present invention accordingly preferably covers at least one of: controlling actuators of the analysis device like a pump drive, temperature control apparatus, and valve actuators; acting on the cartridge or sample; treating the sample; preparing the sample; performing one or more mixing processes and/or reactions with the sample; conveying the sample; and measuring one or more properties of the sample, particularly with the sensor apparatus of the cartridge.

An assay, test or test procedure according to the present invention preferably starts or begins with the analysis device acting on and/or controlling processes on the cartridge and/or the sample. In particular, a test starts or begins with actuators acting on the cartridge. For example, a test can start with conveying the sample within the cartridge.

Methods and/or steps performed before insertion or receiving of the cartridge into/by the analysis device and/or before conveying, treating and/or preparing the sample within said cartridge are preferably not part of an assay, test or test procedure according to the present invention.

The “control information”, thus, preferably is configured to carry out such an assay, test or test procedure or to enable the analysis system or the analysis device to carry out such an assay, test or test procedure. Preferably, said control information is configured to control or to define a control sequence or to be used by the analysis device to carry out said assay, test or test procedure. A “control information”, thus, preferably has instructions being configured for controlling the assay, test or test procedure. In particular, the control information is configured to control an assay, test or test procedure by defining steps or parameters of steps including controlling and/or feedback controlling actuators like the pump drive, the temperature control apparatuses and valve actuators.

The above-mentioned aspects and features of the present invention and the aspects and features of the present invention that will become apparent from the claims and the following description can in principle be implemented independently from one another, but also in any combination or order.

Other aspects, advantages, features and properties of the present invention will become apparent from the claims and the following description of a preferred embodiment with reference to the accompanying drawings.

### BRIEF DESCRIPTION OF THE DRAWINGS

FIG. 1 is a schematic view of a proposed analysis system and/or analysis device comprising a proposed cartridge received therein;

FIG. 2 is a schematic view of the cartridge;

FIG. 3 is a schematic view of the analysis system;

FIG. 4 is a schematic view of a proposed fluid sensor;

FIG. 5 is a schematic section through the proposed fluid sensor;

FIG. 6 is a schematic section through an alternative embodiment of the proposed fluid sensor;

FIG. 7 is a schematic top view of the alternative embodiment according to FIG. 6;

FIG. 8 is a schematic section through another alternative embodiment of the proposed fluid sensor; and

FIG. 9 is a schematic top view of the other alternative embodiment of the proposed fluid sensor.

### DETAILED DESCRIPTION OF THE INVENTION

In the Figures, which are only schematic and sometimes not to scale, the same reference signs are used for the same or similar parts and components, corresponding or comparable properties and advantages being achieved even if these are not repeatedly described.

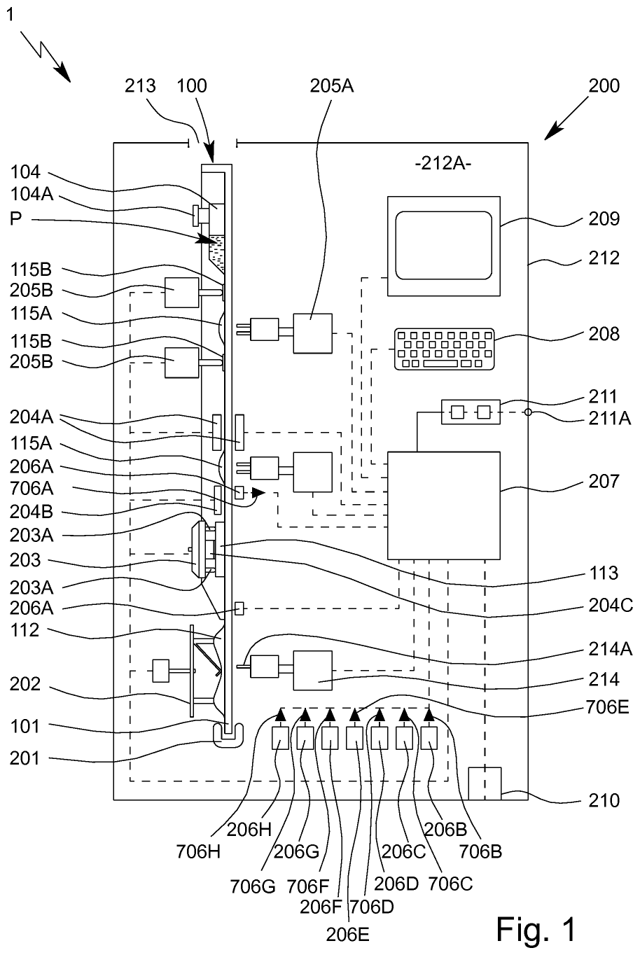

FIG. 1 is a highly schematic view of a proposed analysis system 1 and analysis device 200 for testing an in particular biological sample P, preferably by means of or in an apparatus or cartridge 100.

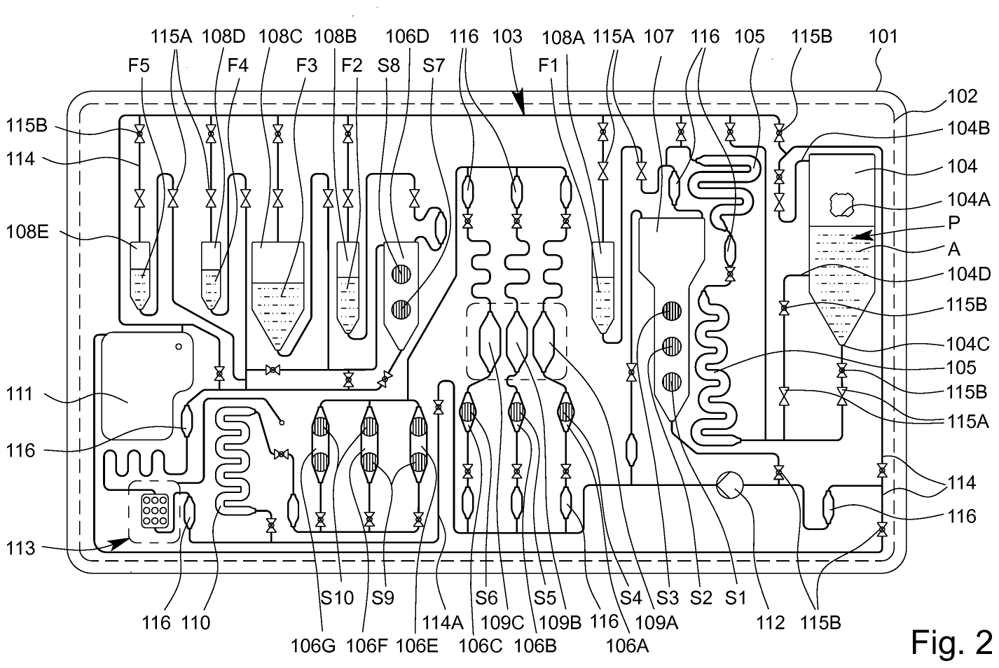

FIG. 2 is a schematic view of a preferred embodiment of the proposed apparatus or cartridge 100 for testing the sample P. The apparatus or cartridge 100 in particular forms a handheld unit, and in the following is merely referred to as a cartridge.

The term “sample” is preferably understood to mean the sample material to be tested, which is in particular taken from a human or animal. In particular, within the meaning of the present invention, a sample is a fluid, such as saliva, blood, urine or another liquid, preferably from a human or animal, or a component thereof. Within the meaning of the present invention, a sample may be pretreated or prepared if necessary, or may come directly from a human or animal or the like, for example. A food sample, environmental sample or another sample may optionally also be tested, in particular for environmental analytics, food safety and/or for detecting other substances, preferably natural substances, but also biological or chemical warfare agents, poisons or the like.

Preferably, the analysis system 1 and/or analysis device 200 controls the testing of the sample P in particular in or on the cartridge 100 and/or is used to evaluate the testing and/or to collect, to process and/or to store measured values from the test.

The analysis system 1 preferably comprises one or more cartridges 100 for receiving the sample P. The analysis system 1 preferably comprises the analysis device 200 for receiving the cartridge 100 and subsequently carrying out the test using the received cartridge 100.

By means of the proposed analysis system 1, analysis device 200 and/or the cartridge 100 and/or using the proposed method for testing the sample P, preferably an analyte A of the sample P, in particular a (certain) nucleic-acid sequence and/or a (certain) protein, or particularly preferably a plurality of analytes A of the sample P, can be determined, identified or detected. Said analytes A are in particular detected, identified and/or measured not only qualitatively, but particularly preferably also quantitatively.

Therefore, the sample P can in particular be tested for qualitatively or quantitatively determining at least one analyte A, for example in order for it to be possible to detect a disease and/or pathogen or to determine other values, which are important for diagnostics, for example.

Particularly preferably, a molecular-biological test is made possible by means of the analysis system 1 and/or analysis device 200 and/or by means of the cartridge 100.

Particularly preferably, a nucleic-acid assay for detecting a nucleic-acid sequence, in particular a DNA sequence and/or RNA sequence, and/or a protein assay for detecting a protein, in particular an antigen and/or antibody, are made possible or are carried out.

Preferably, the sample P or individual components of the sample P or analyte A can be amplified if necessary, in particular by means of PCR, and tested, identified or detected in the analysis system 1, analysis device 200 and/or in the cartridge 100, and/or for the purpose of carrying out the nucleic-acid assay. Preferably, amplification products of the analyte A or analytes A are thus produced.

In the following, further details are first given on a preferred construction of the cartridge 100, with features of the cartridge 100 preferably also directly representing features of the analysis system 1, in particular even without any further explicit explanation.

The cartridge 100 is preferably at least substantially planar, flat, plate-shaped and/or card-like.

The cartridge 100 preferably comprises an in particular at least substantially planar, flat, plate-shaped and/or card-like main body or support 101, the main body or support 101 in particular being made of and/or injection-molded from plastics material, particularly preferably polypropylene.

The cartridge 100 preferably comprises at least one film or cover 102 for covering the main body 101 and/or cavities and/or channels formed therein at least in part, in particular on the front, and/or for forming valves or the like, as shown by dashed lines in FIG. 2.

The analysis system 1 or cartridge 100 or the main body 101 thereof, in particular together with the cover 102, preferably forms and/or comprises a fluidic system 103, referred to in the following as the fluid system 103.

The cartridge 100, the main body 101 and/or the fluid system 103 are preferably at least substantially vertically oriented in the operating position and/or during the test, in particular in the analysis device 200, as shown schematically in FIG. 1. In particular, the main plane or surface extension of the cartridge 100 thus extends at least substantially vertically in the operating position.

The cartridge 100 and/or the fluid system 103 preferably comprises a plurality of cavities, in particular at least one receiving cavity 104, at least one metering cavity 105, at least one intermediate cavity 106A-G, at least one mixing cavity 107, at least one storage cavity 108, at least one reaction cavity 109A-C, at least one intermediate temperature-control cavity 110 and/or at least one collection cavity 111, as shown in FIG. 1 and FIG. 2.

The cartridge 100 and/or the fluid system 103 also preferably comprises at least one pump apparatus 112 and/or at least one sensor arrangement or sensor apparatus 113.

Some, most or all of the cavities are preferably formed by chambers and/or channels or other depressions in the cartridge 100 and/or the main body 101, and particularly preferably are covered or closed by the cover 102. However, other structural solutions are also possible.

In the example shown, the cartridge 100 or the fluid system 103 preferably comprises two metering cavities 105, a plurality of intermediate cavities 106A to 106G, a plurality of storage cavities 108A to 108E and/or a plurality of reaction cavities 109A-C, which can preferably be loaded separately from one another, in particular a first reaction cavity 109A, a second reaction cavity 109B and an optional third reaction cavity 109C, as can be seen in FIG. 2.

The reaction cavity/cavities 109A-C is/are used in particular to carry out an amplification reaction, in particular PCR, or several, preferably different, amplification reactions, in particular PCRs. It is preferable to carry out several, preferably different, PCRs, i.e. PCRs having different primer combinations or primer pairs, in parallel and/or independently and/or in different reaction cavities 109A-C.

To carry out the nucleic-acid assay, preferably nucleic-acid sequences, as analytes A of the sample P, are amplified in the reaction cavity/cavities 109A-C by means of an amplification reaction, in particular in order to produce amplification products for the subsequent detection in the sensor arrangement or sensor apparatus 113.

Within the meaning of the present invention, amplification reactions are in particular molecular-biological reactions in which an analyte A, in particular a nucleic-acid sequence, is amplified/copied and/or in which amplification products, in particular nucleic-acid products, of an analyte A are produced. Particularly preferably, PCRs are amplification reactions within the meaning of the present invention.

The amplification products V and/or other portions of the sample P produced in the one or more reaction cavities 109A-C can be conducted or fed to the connected sensor arrangement or sensor apparatus 113, in particular by means of the pump apparatus 112.

The sensor apparatus 113 is used in particular for detecting, particularly preferably qualitatively and/or quantitatively determining, the analyte A or analytes A of the sample P, in this case particularly preferably the nucleic-acid sequences and/or proteins as the analytes A. Alternatively or additionally, however, other values may also be collected or determined.

As already explained at the outset, in particular nucleic-acid sequences, preferably DNA sequences and/or RNA sequences, and/or proteins, in particular antigens and/or antibodies, are preferably qualitatively and/or quantitatively determined as analytes A of the sample P. In the following, however, a distinction is not made between nucleic-acid sequences and proteins, or between the nucleic-acid assay for detecting nucleic-acid sequences and the protein assay for detecting proteins.

In particular, the pump apparatus 112 comprises or forms a tube-like or bead-like raised portion, in particular by means of the film or cover 102, particularly preferably on the back of the cartridge 100, as shown schematically in FIG. 1.

The cartridge 100, the main body 101 and/or the fluid system 103 preferably comprise a plurality of channels 114 and/or valves 115A, 115B, as shown in FIG. 2.

By means of the channels 114 and/or valves 115A, 115B, the cavities 104 to 111, the pump apparatus 112 and/or the sensor arrangement and/or sensor apparatus 113 can be temporarily and/or permanently fluidically interconnected and/or fluidically separated from one another, as required and/or optionally or selectively, in particular such that they are controlled by the analysis system 1 or the analysis device 200.

The cavities 104 to 111 are preferably each fluidically linked or interconnected by a plurality of channels 114. Particularly preferably, each cavity is linked or connected by at least two associated channels 114, in order to make it possible for fluid to fill, flow through and/or drain from the respective cavities as required.

The fluid transport or the fluid system 103 is preferably not based on capillary forces, or is not exclusively based on said forces, but in particular is essentially based on the effects of gravity and/or pumping forces and/or compressive forces and/or suction forces that arise, which are particularly preferably generated by the pump or pump apparatus 112. In this case, the flows of fluid or the fluid transport and the metering are controlled by accordingly opening and closing the valves 115A, 115B and/or by accordingly operating the pump or pump apparatus 112, in particular by means of a pump drive 202 of the analysis device 200.

Preferably, each of the cavities 104 to 110 has an inlet at the top and an outlet at the bottom in the operating position. Therefore, if required, only liquid from the respective cavities can be removed via the outlet.

In the operating position, the liquids from the respective cavities are preferably removed, in particular drawn out, via the outlet that is at the bottom in each case, it preferably being possible for gas or air to flow and/or be pumped into the respective cavities via the inlet that is in particular at the top. In particular, relevant vacuums in the cavities can thus be prevented or at least minimized when conveying the liquids.

In particular, the cavities, particularly preferably the storage cavity/cavities 108, the mixing cavity 107 and/or the receiving cavity 104, are each dimensioned and/or oriented in the normal operating position such that, when said cavities are filled with liquid, bubbles of gas or air that may potentially form rise upwards in the operating position, such that the liquid collects above the outlet without bubbles. However, other solutions are also possible here.

The receiving cavity 104 preferably comprises a connection 104A for introducing the sample P. In particular, the sample P may for example be introduced into the receiving cavity 104 and/or cartridge 100 via the connection 104A by means of a pipette, syringe or other instrument.

The receiving cavity 104 preferably comprises an inlet 104B, an outlet 104C and an optional intermediate connection 104D, it preferably being possible for the sample P or a portion thereof to be removed and/or conveyed further via the outlet 104C and/or the optional intermediate connection 104D. Gas, air or another fluid can flow in and/or be pumped in via the inlet 104B, as already explained.

Preferably, the sample P or a portion thereof can be removed, optionally and/or depending on the assay to be carried out, via the outlet 104C or the optional intermediate connection 104D of the receiving cavity 104. In particular, a supernatant of the sample P, such as blood plasma or blood serum, can be conducted away or removed via the optional intermediate connection 104D, in particular for carrying out the protein assay.

Preferably, at least one valve 115A, 115B is assigned to each cavity, the pump apparatus 112 and/or the sensor apparatus 113 and/or is arranged upstream of the respective inlets and/or downstream of the respective outlets.

Preferably, the cavities 104 to 111 or sequences of cavities 104 to 111, through which fluid flows in series or in succession for example, can be selectively released and/or fluid can selectively flow therethrough by the assigned valves 115A, 115B being actuated, and/or said cavities can be fluidically connected to the fluid system 103 and/or to other cavities.

In particular, the valves 115A, 115B are formed by the main body 101 and the film or cover 102 and/or are formed in another manner, for example by additional layers, depressions or the like.

Particularly preferably, one or more valves 115A are provided which are preferably tightly closed initially or in the storage state, particularly preferably in order to seal liquids or liquid reagents F, located in the storage cavities 108, and/or the fluid system 103 from the open receiving cavity 104 in a storage-stable manner.

Preferably, an initially closed valve 115A is arranged upstream and downstream of each storage cavity 108. Said valves are preferably only opened, in particular automatically, when the cartridge 100 is actually being used and/or while inserting the cartridge 100 into the analysis device 200 and/or for carrying out the assay.

A plurality of valves 115A, in particular three valves in this case, are preferably assigned to the receiving cavity 104, in particular if the intermediate connection 104D is provided in addition to the inlet 104B and the outlet 104C. Depending on the use, in addition to the valve 115A on the inlet 104B, then preferably only the valve 115A either at the outlet 104C or at the intermediate connection 104D is opened.

The valves 115A assigned to the receiving cavity 104 seal the fluid system 103 and/or the cartridge 100 in particular fluidically and/or in a gas-tight manner until the sample P is inserted and the receiving cavity 104 or a connection 104A of the receiving cavity 104 is closed.

As an alternative or in addition to the valves 115A (which are initially closed), one or more valves 115B are preferably provided which are not closed in a storage-stable manner and/or which are open initially and/or which can be closed by actuation. These valves are used in particular to control the flows of fluid during the test.

The cartridge 100 is preferably designed as a microfluidic card and/or the fluid system 103 is preferably designed as a microfluidic system. In the present invention, the term “microfluidic” is preferably understood to mean that the respective volumes of individual cavities, some of the cavities or all of the cavities 104 to 111 and/or channels 114 are, separately or cumulatively, less than 5 ml or 2 ml, particularly preferably less than 1 ml or 800 μl, in particular less than 600 μl or 300 μl, more particularly preferably less than 200 μl or 100 μl.

Particularly preferably, a sample P having a maximum volume of 5 ml, 2 ml or 1 ml can be introduced into the cartridge 100 and/or the fluid system 103, in particular the receiving cavity 104.

Reagents and liquids which are preferably introduced or provided before the test in liquid form as liquids or liquid reagents F and/or in dry form as dry reagents S are required for testing the sample P, as shown in the schematic view according to FIG. 2 by reference signs F1 to F5 and S1 to S10.

Furthermore, other liquids F, in particular in the form of a wash buffer, solvent for dry reagents S and/or a substrate, for example in order to form detection molecules and/or a redox system, are also preferably required for the test, the detection process and/or for other purposes, and are in particular provided in the cartridge 100, i.e. are likewise introduced before use, in particular before delivery. At some points in the following, a distinction is not made between liquid reagents and other liquids, and therefore the respective explanations are accordingly also mutually applicable.

The analysis system 1 or the cartridge 100 preferably contains all the reagents and liquids required for pretreating the sample P and/or for carrying out the test or assay, in particular for carrying out one or more amplification reactions or PCRs, and therefore, particularly preferably, it is only necessary to receive the optionally pretreated sample P.

The cartridge 100 or the fluid system 103 preferably comprises a bypass 114A that can optionally be used, in order for it to be possible, if necessary, to conduct or convey the sample P or components thereof past the reaction cavities 109A-C and/or, by bypassing the optional intermediate temperature-control cavity 110, also directly to the sensor apparatus 113.

The cartridge 100, the fluid system 103 and/or the channels 114 preferably comprise sensor portions 116 or other apparatuses for detecting liquid fronts and/or flows of fluid.

It is noted that various components, such as the channels 114, the valves 115A, 115B, in particular the valves 115A that are initially closed and the valves 115B that are initially open, and the sensor portions 116 in FIG. 2 are, for reasons of clarity, only labelled in some cases, but the same symbols are used in FIG. 2 for each of these components.

The collection cavity 111 is preferably used for receiving excess or used reagents and liquids and volumes of the sample, and/or for providing gas or air in order to empty individual cavities and/or channels.

In particular, the collection cavity 111 can optionally be connected to individual cavities and channels or other apparatuses fluidically in order to remove reagents and liquids from said cavities, channels or other apparatuses and/or to replace said reagents and liquids with gas or air. The collection cavity 111 is preferably given appropriate large dimensions.

Once the sample P has been introduced into the receiving cavity 104 and the connection 104A has been closed, the cartridge 100 can be inserted into and/or received in the proposed analysis device 200 in order to test the sample P, as shown in FIG. 1. Alternatively, the sample P could also be fed in later.

FIG. 1 shows the analysis system 1 in a ready-to-use state for carrying out a test or assay on the sample P received in the cartridge 100. In this state, the cartridge 100 is therefore linked to, received by and/or inserted into the analysis device 200.

In the following, some features and aspects of the analysis device 200 are first explained in greater detail, in particular on the basis of FIG. 1. The features and aspects relating to said device are preferably also directly features and aspects of the proposed analysis system 1, in particular even without any further explicit explanation.

The analysis system 1 or analysis device 200 preferably comprises a mount or receptacle 201 for mounting and/or receiving the cartridge 100.

Preferably, the cartridge 100 is fluidically, in particular hydraulically, separated or isolated from the analysis device 200. In particular, the cartridge 100 forms a preferably independent and in particular closed or sealed fluidic or hydraulic system 103 for the sample P and the reagents and other liquids. In this way, the analysis device 200 does not come into direct contact with the sample P and can in particular be reused for another test without being disinfected and/or cleaned first.

It is however provided that the analysis device 200 can be connected or coupled mechanically, electrically, thermally and/or pneumatically to the cartridge 100.

In particular, the analysis device 200 is designed to have a mechanical effect, in particular for actuating the pump apparatus 112 and/or the valves 115A, 115B, and/or to have a thermal effect, in particular for temperature-controlling the reaction cavity/cavities 109A-C and/or the intermediate temperature-control cavity 110.

In addition, the analysis device 200 can preferably be pneumatically connected to the cartridge 100, in particular in order to actuate individual apparatuses, and/or can be electrically connected to the cartridge 100, in particular in order to collect and/or transmit measured values, for example from the sensor apparatus 113 and/or sensor portions 116.

The analysis system 1 or analysis device 200 preferably comprises a pump drive 202, the pump drive 202 in particular being designed for mechanically actuating the pump apparatus 112.

Preferably, a head of the pump drive 202 can be rotated in order to rotationally axially depress the preferably bead-like raised portion of the pump apparatus 112. Particularly preferably, the pump drive 202 and pump apparatus 112 together form a pump, in particular in the manner of a hose pump or peristaltic pump and/or a metering pump, for the fluid system 103 and/or the cartridge 100.

Particularly preferably, the pump is constructed as described in German Patent No. DE 10 2011 015 184 B4 and corresponding U.S. Pat. No. 8,950,424 B2. However, other structural solutions are also possible.

Preferably, the capacity and/or discharge rate of the pump can be controlled and/or the conveying direction of the pump and/or pump drive 202 can be switched. Preferably, fluid can thus be pumped forwards or backwards as desired.

The analysis system 1 or analysis device 200 preferably comprises a connection apparatus 203 for in particular electrically and/or thermally connecting the cartridge 100 and/or the sensor arrangement or sensor apparatus 113.

As shown in FIG. 1, the connection apparatus 203 preferably comprises a plurality of electrical contact elements 203A, the cartridge 100, in particular the sensor arrangement or sensor apparatus 113, preferably being electrically connected or connectable to the analysis device 200 by the contact elements 203A.

The analysis system 1 or analysis device 200 preferably comprises one or more temperature-control apparatuses for temperature-controlling the cartridge 100 and/or having a thermal effect on the cartridge 100, in particular for heating and/or cooling, the temperature-control apparatus(es) (each) preferably comprising or being formed by a heating resistor or a Peltier element.

Individual temperature-control apparatuses, some of these apparatuses or all of these apparatuses can preferably be positioned against or abutted on the cartridge 100, the main body 101, the cover 102, the sensor arrangement, sensor apparatus 113 and/or individual cavities and/or can be thermally coupled thereto and/or can be integrated therein and/or in particular can be operated or controlled electrically by the analysis device 200. In the example shown, in particular the temperature-control apparatuses 204A-C are provided.

Preferably, the temperature-control apparatus 204A, referred to in the following as the reaction temperature-control apparatus 204A, is assigned to one of the reaction cavities 109A-C or to a plurality of reaction cavities 109A-C, in particular in order for it to be possible to carry out one or more amplification reactions therein.

The reaction cavities 109A-C are preferably temperature-controlled simultaneously and/or uniformly, in particular by means of one common reaction temperature-control apparatus 204A or two reaction temperature-control apparatuses 204A.

More particularly preferably, the reaction cavity/cavities 109A-C can be temperature-controlled from two different sides and/or by means of two or the reaction temperature-control apparatuses 204A that are preferably arranged on opposite sides.

Alternatively, each reaction cavity 109A-C can be temperature-controlled independently and/or individually.

The temperature-control apparatus 204B, referred to in the following as the intermediate temperature-control apparatus 204B, is preferably assigned to the intermediate temperature-control cavity 110 and/or is designed to (actively) temperature-control or heat the intermediate temperature-control cavity 110 and/or a fluid located therein, in particular the amplification products, preferably to a preheat temperature.

The intermediate temperature-control cavity 110 and/or intermediate temperature-control apparatus 204B is preferably arranged upstream of or (immediately) before the sensor arrangement or sensor apparatus 113, in particular in order for it to be possible to temperature-control or preheat, in a desired manner, fluids to be fed to the sensor arrangement or sensor apparatus 113, in particular analytes A and/or amplification products, particularly preferably immediately before said fluids are fed.

Particularly preferably, the intermediate temperature-control cavity 110 or intermediate temperature-control apparatus 204B is designed or provided to denature the sample P or analytes A and/or the amplification products V produced, and/or to divide any double-stranded analytes A or amplification products into single strands and/or to counteract premature bonding or hybridizing of the amplification products V, in particular by the addition of heat.

Preferably, the analysis system 1, analysis device 200 and/or the cartridge 100 and/or one or each temperature-control apparatus comprise/comprises a temperature detector and/or temperature sensor (not shown), in particular in order to make it possible to control and/or feedback control temperature.

One or more temperature sensors may for example be assigned to the sensor portions 116 and/or to individual channel portions or cavities, i.e. may be thermally coupled thereto.

The temperature-control apparatus 204C, referred to in the following as the sensor temperature-control apparatus 204C, is in particular assigned to the sensor apparatus 113 and/or is designed to (actively) temperature-control or heat fluids located in or on the sensor arrangement or sensor apparatus 113, in particular analytes A and/or amplification products, reagents or the like, in a desired manner, preferably to a hybridization temperature.

The sensor temperature-control apparatus 204C is preferably planar and/or has a contact surface which is preferably rectangular and/or corresponds to the dimensions of the sensor arrangement or sensor apparatus 113, the contact surface allowing for heat transfer between the sensor temperature-control apparatus 204C and the sensor apparatus 113.

Preferably, the analysis device 200 comprises the sensor temperature-control apparatus 204C. However, other structural solutions are also possible in which the sensor temperature-control apparatus 204C is integrated in the cartridge 100, in particular the sensor arrangement or sensor apparatus 113.

Particularly preferably, the connection apparatus 203 comprises the sensor temperature-control apparatus 204C, and/or the connection apparatus 203 together with the sensor temperature-control apparatus 204C can be linked to, in particular pressed against, the cartridge 100, in particular the sensor arrangement or sensor apparatus 113.

More particularly preferably, the connection apparatus 203 and the sensor temperature-control apparatus 204C (together) can be moved towards and/or relative to the cartridge 100, in particular the sensor arrangement or sensor apparatus 113, and/or can be positioned against said cartridge, preferably in order to both electrically and thermally couple the analysis device 200 to the cartridge 100, in particular the sensor arrangement or sensor apparatus 113 or the support thereof.

Preferably, the sensor temperature-control apparatus 204C is arranged centrally on the connection apparatus 203 or a support thereof and/or is arranged between the contact elements 203A.

In particular, the contact elements 203A are arranged in an edge region of the connection apparatus 203 or a support thereof or are arranged around the sensor temperature-control apparatus 204C, preferably such that the connection apparatus 203 is connected or connectable to the sensor apparatus 113 thermally in the center and electrically on the outside or in the edge region. However, other solutions are also possible here.

The analysis system 1 or analysis device 200 preferably comprises one or more valve actuators 205A, B for actuating the valves 115A, 115B. Particularly preferably, different (types or groups of) valve actuators 205A and 205B are provided which are assigned to the different (types or groups of) valves 115A and 115B for actuating each of said valves, respectively.

The analysis system 1 or analysis device 200 preferably comprises a control apparatus 207 for controlling the sequence of a test or assay and/or for collecting, evaluating and/or outputting or providing measured values in particular from the sensor apparatus 113, and/or test results and/or other data or values.

The control apparatus 207 preferably comprises an internal clock or time base by means of which the sequence of the test is or can be controlled and/or by means of which test steps that follows temporally one another or that extend over time are controlled or can be controlled by the control apparatus 207.

The control apparatus 207 preferably controls or is designed to control actuators of the analysis device 200 for acting on the cartridge 100 in order to carry out the test. The actuators are in particular the pump drive 202, the temperature-control apparatuses and/or the valve actuators 205A, B.

The analysis system 1 or analysis device 200 preferably comprises one or more sensors 206A-H.

In one aspect of the present invention, which can also be implemented independently, one or more fluid sensors 206A are designed, provided or intended to detect liquid fronts PF1, PF2 and/or flows of fluid in the fluid system 103.

Particularly preferably, the fluid sensors 206A are designed to measure or detect, for example optically and/or capacitively, a liquid front PF1, PF2 and/or the presence, the speed, the mass flow rate/volume flow rate, the temperature and/or another value of a fluid in a channel and/or a cavity, in particular in a respectively assigned sensor portion 116, which is in particular formed by a planar and/or widened channel portion of the fluid system 103.

The fluid sensor/fluid sensors 206A preferably measures/measure a fluid or a liquid entering or leaving the sensor portion 116 and/or a content change or fluid change in the sensor portion 116, and in the process generates a measurement result 706A that corresponds to the fluid entering, the fluid leaving, the content change and/or the fluid change in the sensor portion 116.

This measurement result 706A from the fluid sensor 206A can be retrieved by the control apparatus 207 and/or transmitted to the control apparatus 207. The control apparatus 207 controls or is designed to control the test and/or the actuators, preferably using or taking into account the measurement result 706A from the fluid sensor 206A.

In particular, when a content change, an entering fluid, a leaving fluid and/or a fluid change is detected in the sensor portion 116, in particular when a liquid front PF1, PF2 is detected, the control apparatus 207 influences a program sequence. In this case, for example a control can be carried out or a subsequent step of the test can be controlled, in particular by activating the actuators in a particular and/or differing manner.

Particularly preferably, the sensor portions 116 are each oriented and/or incorporated in the fluid system 103 and/or fluid flows against or through the sensor portions 116 such that, in the operating position of the cartridge 100, fluid flows through the sensor portions 116 in the vertical direction and/or from the bottom to the top, or vice versa, in particular in order to make it possible or easier to accurately detect liquid.

Alternatively or additionally, the analysis device 200 preferably comprises one or more (different, other and/or further) sensors 206B-206H which preferably generate or are designed to generate measurement results 706 B-H.

The sensor 206B can be a pressure sensor for determining the (relative) air pressure.

Alternatively or additionally, one or more temperature sensors 206C are provided for detecting the internal temperature and/or the temperature in the interior space 212A of the analysis device 200, in particular the temperature of an atmosphere in the interior space 212A.

Alternatively or additionally, one or more temperature sensors 206C are provided for detecting the ambient temperature and/or the temperature of an atmosphere surrounding the analysis device 200 and/or the temperature of one or more of the temperature-control apparatuses.

The analysis device 200 preferably comprises a tilt sensor 206D for detecting the inclination and/or orientation of the analysis device 200 and/or of the cartridge 100.

The analysis device 200 may comprise an acceleration sensor 206E. The acceleration sensor 206E is preferably designed to determine an acceleration of the analysis device 200, in particular an acceleration in the vertical and/or horizontal direction with respect to the operating position.

The analysis device 200 may comprise a humidity sensor 206F for determining the (relative) atmospheric humidity and/or the dew point of the atmosphere inside or in the interior space 212A and/or outside the analysis device 200.

The analysis device 200 may comprise a position sensor 206G for determining the position or location, for example by means of a GPS sensor. The position sensor 206G is preferably designed to determine the location of the analysis device in space, in particular on the Earth's surface, and/or to output the geographical position, the location and/or the coordinates of the analysis device 200.

The analysis device 200 may comprise a cartridge sensor 206H for determining or checking the position or alignment of the cartridge 100 in or with respect to the analysis device 200.

The control apparatus 207 controls or is designed to control the test and/or the actuators, preferably using or taking into account the measurement results 706A-H from the sensors 206A-H. In this case, the control apparatus 207 preferably controls or feedback controls actuators such that they act on the cartridge 100 in order to carry out the test. In particular, the control apparatus 207 controls the pump drive 202, the temperature-control apparatuses 204 and/or valve actuators 205, in particular taking into account or depending on one or more of the measured values 706A-H from the sensors 206 A-H.

The flows of fluid are controlled in particular by accordingly activating the pump or pump apparatus 112 and actuating the valves 115A, 115B. Particularly preferably, the pump drive 202 comprises a stepper motor, or a drive calibrated in another way, such that desired metering can be achieved, at least in principle, by means of appropriate activation.

Additionally or alternatively, the fluid sensors 206A are used to detect liquid fronts PF1, PF2 or flows of fluid, in particular in cooperation with the assigned sensor portions 116, in order to achieve the desired fluidic sequence and the desired metering by accordingly controlling the pump or pump apparatus 112 and accordingly activating the valves 115A, 115B.

Optionally, the analysis system 1 or analysis device 200 comprises an input apparatus 208, such as a keyboard, a touch screen or the like, and/or a display apparatus 209, such as a screen.

The analysis system 1 or analysis device 200 preferably comprises at least one interface 210, for example for controlling, for communicating and/or for outputting measured data or test results and/or for linking to other devices, such as a printer, an external power supply or the like. This may in particular be a wired or wireless interface 210.

The analysis system 1 or analysis device 200 preferably comprises a power supply 211, preferably a battery or an accumulator, which is in particular integrated and/or externally connected or connectable. Preferably, an integrated accumulator is provided as a power supply 211 and can be (re)charged by an external charging device (not shown) via a connection 211A and/or is interchangeable.

The analysis system 1 or analysis device 200 preferably comprises a housing 212, all the components and/or some or all of the apparatuses preferably being integrated in the housing 212. Particularly preferably, the cartridge 100 can be inserted or slid into the housing 212, and/or can be received by the analysis device 200, through an opening 213 which can in particular be closed, such as a slot or the like.

The analysis system 1 or analysis device 200 is preferably portable or mobile. Particularly preferably, the analysis device 200 weighs less than 25 kg or 20 kg, particularly preferably less than 15 kg or 10 kg, in particular less than 9 kg or 6 kg.

The fluidic, in particular pneumatic, coupling between the cartridge 100 and the analysis device 200 will be explained in greater detail in the following, it being possible for the following aspects to be implemented independently from the preceding aspects.

As already explained, the analysis device 200 can preferably be pneumatically linked to the cartridge 100, in particular to the sensor arrangement or sensor apparatus 113 and/or to the pump apparatus 112.

Particularly preferably, the analysis device 200 is designed to supply the cartridge 100, in particular the sensor arrangement or sensor apparatus 113 and/or the pump apparatus 112, with a working medium, in particular gas or air.

Preferably, the working medium can be compressed and/or pressurised in the analysis device 200 or by means of the analysis device 200.

Preferably, the analysis device 200 comprises a pressurised gas supply 214 for this purpose, in particular a pressure generator or compressor, preferably in order to compress and/or pressurise the working medium.

The pressurised gas supply 214 is preferably integrated in the analysis device 200 or the housing 212 and/or can be controlled or feedback controlled by means of the control apparatus 207. The pressurised gas supply 214 can also, at least in part, be formed on or by the cartridge 100.

Preferably, the pressurised gas supply 214 is electrically operated or can be operated by electrical power. In particular, the pressurised gas supply 214 can be supplied with electrical power by means of the power supply 211.

The analysis device 200 or pressurised gas supply 214 is preferably designed to compress the working medium to a pressure of more than 100 kPa, particularly preferably more than 150 kPa or 250 kPa, in particular more than 300 kPa or 350 kPa, and/or of less than 1 MPa, particularly preferably less than 900 kPa or 800 kPa, in particular less than 700 kPa and/or to feed said medium to the cartridge 100 at said pressure.

Preferably, air can be drawn in, in particular from the surroundings, as the working medium by means of the analysis device 200 or pressurised gas supply 214. In particular, the analysis device 200 or pressurised gas supply 214 is designed to use the surroundings as a reservoir for the working medium or the air. However, other solutions are also possible here, in particular those in which the analysis device 200 or pressurised gas supply 214 comprises a preferably closed or delimited reservoir, such as a tank or container, comprising the working medium, and/or is connected or connectable thereto.

Preferably, the analysis device 200 or pressurised gas supply 214 comprises an inlet, the working medium in particular being able to be drawn in and/or conducted in the pressurised gas supply 214 via the inlet.

Preferably, the analysis device 200 or pressurised gas supply 214 comprises a filter, the filter preferably being integrated in the inlet and/or it preferably being possible for the working medium to be filtered by means of the filter and/or it preferably being possible for particles to be separated from the working medium by means of the filter.

The filter is preferably designed as a micro filter or as a fine particulate air filter. Preferably, particles having a particle diameter of more than 10 μm, particularly preferably more than 8 μm or 9 μm, in particular more than 6 μm or 7 μm, more particularly preferably more than 4 μm or 5 μm, can be separated by means of the filter, the particle diameter preferably being the maximum or average diameter of the respective particles. This ensures that the channels or lines in the cartridge that convey the working medium do not become contaminated or clogged and/or that no undesired pressure loss occurs.

The analysis device 200 or pressurised gas supply 214 preferably comprises a connection element 214A, in particular in order to pneumatically connect the analysis device 200 and/or pressurised gas supply 214 to the cartridge 100.

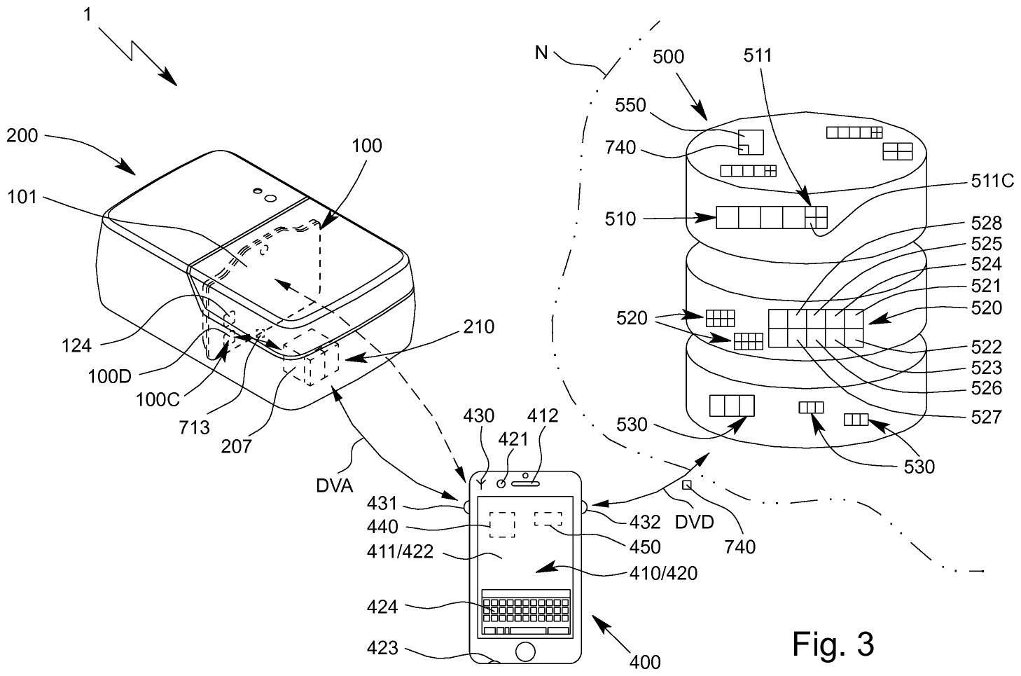

FIG. 3 is a schematic view of the proposed analysis system 1 for testing an in particular biological sample P, comprising the analysis device 200 for receiving the cartridge 100 and subsequently carrying out the test using the received cartridge 100, and an operating instrument 400 for the analysis device 200.

The operating instrument 400 is preferably designed to control the analysis device 200. Alternatively or additionally, the operating instrument 400 can receive or retrieve information, in particular (measurement) results such as measured values, from the analysis device 200. In particular, the operating instrument 400 is a mobile terminal device such as a smartphone, a tablet or the like.

The operating instrument 400 is preferably implemented or provided so as to be physically separated from the analysis device 200. The operating instrument 400 can preferably be separated and/or disconnected from the analysis device 200 physically and/or with respect to a data connection.

The operating instrument 400 can preferably be wirelessly connected to the analysis device 200. A data connection DVA can thus be established between the analysis device 200 and the operating instrument 400. However, the data connection DVA can in principle also be established in another manner, for example wired.

It is preferable for the operating instrument 400 to also be operational when separated or disconnected from the analysis device 200, in particular for carrying out evaluations or for other purposes. Alternatively or additionally, the analysis device 200 is also operational when separated or disconnected from the operating instrument 400, in particular for continuing a test.

Particularly preferably, the operating instrument 400 comprises an interface 430 for establishing data connections DVA, DVD. The interface 430 and/or the operating instrument 400 in particular comprises what is referred to as an analysis device interface 431 that is designed to establish the preferably wireless data connection DVA to the analysis device 200. This can, for example, be a radio interface, WPAN interface, Bluetooth interface and/or a Bluetooth module or the like.

The interface 210 of the analysis device 200 preferably corresponds to the interface 430 and/or the analysis device interface 431 of the operating instrument 400, in particular such that the data connection DVA between the operating instrument 400 and the analysis device 200 can be established. The interface 210 of the analysis device 200 and the analysis device interface 431 preferably support the same data transmission method and/or radio transmission method or radio standard, in particular WLAN or WPAN methods such as Bluetooth, NFC, Zigbee or the like.

Particularly preferably, the interface 210 of the analysis device 200 and the analysis device interface 431 make possible or facilitate what is known as an ad-hoc connection. In this case, the data connection DVA is established preferably automatically when the devices, i.e. the operating instrument 400 and the analysis device 200, are within range of one another.

In order to control the test, it is preferable for precisely one data connection DVA to be provided between the analysis device 200 to be controlled and the operating instrument 400 controlling the analysis device 200, and/or for control information 510 to be received and/or accepted or to be acceptable and/or receivable and/or for measurement results 713 to be transmitted or to be transmittable only via precisely one data connection DVA between the analysis device 200 to be controlled and the operating instrument 400 controlling the analysis device 200.

The analysis system 1 preferably further comprises a database 500 or the database 500 is assigned to the analysis system 1. The database 500 is preferably an external database 500 that is implemented or provided so as to be physically separated from the operating instrument 400 and/or from the analysis device 200. In principle, however, it is not impossible for the database 500 to be provided or implemented such that it can be directly linked, in particular to the operating instrument 400, or to be provided or implemented by the operating instrument 400.

The operating instrument 400 can access the database 500 via a data connection DVD. For this purpose, the operating instrument 400 and/or the interface 430 can comprise a database interface 432 by means of which the database 500 can be accessed, in particular via a network N. The network N may be the Internet or another data network N. It is also preferable for the operating instrument 400 to be able to establish the data connection DVD to the database 500 via a wireless interface, in particular WLAN, WPAN, mobile communications or the like. However, in principle, other solutions are also possible here.

The analysis system 1, in particular the database 500, preferably comprises control information 510 by means of which the analysis device 200 can be controlled in order to carry out a test.

The control information 510 preferably defines the actuation of the actuators of the analysis device 200 in a particular manner, such that the sample P is tested in the cartridge 100. In particular, actuators for carrying out the test can be or are controlled using the control information 510 such that said actuators act on the cartridge 100 and/or the sample P. These actuators are in particular the pump drive 202 and/or one or more temperature-control apparatuses 204 and/or one or more valve actuators 205. The control information 510 preferably comprises parameters and/or instructions for carrying out one or more steps of the method for testing the sample P explained above.

Alternatively or additionally, the control information 510 comprises execution information 511 for executing the test, in particular the sequence for controlling different actuators. The execution information 511 can also be separate from the control information 510, can be stored in the database 500 and/or can be transmitted to the analysis device 200 and/or the operating instrument 400.

Preferably, the analysis system 1 comprises calibration information 520 that can be stored in the database 500 and/or can be retrieved from the database 500. The calibration information 520 is preferably capable of influencing the test of the sample P, in particular depending on the specific cartridge 100, on a cartridge batch of the specific cartridge 100 and/or on the specific test.

The calibration information 520 is in particular default or basic settings, parameters and/or threshold values for sensors such as the sensor apparatus 113 of the cartridge 100, for one or more of the sensor(s) 206A-H of the analysis device 200 and/or for one or more of the actuators.

Calibration information 520 can be used in addition to control information 510 for carrying out the test, the calibration information 520 preferably influencing or specifying the control information 510. The calibration information 520 can be or can form the control information 510 or a part of the control information 510, even if this is not explicitly mentioned in the following.

The analysis device 200 can be calibrated and/or configured by calibration information 520 that can form part of the control information 510 or can be provided separately. For this purpose, the calibration information 520 can be determined, retrieved and/or transmitted to the analysis device 200 by means of the operating instrument 400.

In one example, fluid sensor calibration information 521 is provided which influences setting and/or evaluation of the fluid sensor 206A. The fluid sensor calibration information 521 is preferably dependent on the test to be carried out, the phase of the test and/or expected effects of a content change in a sensor portion 116 on the fluid sensor 206A during the test sequence, and/or contains various specifications which are dependent thereon.

Alternatively or additionally, tilt sensor calibration information 524 can be provided, preferably one or more threshold values 525, in particular a start threshold value 526 for blocking the start of a test if said threshold value is exceeded, and/or an interruption threshold value 527 for interrupting the test and/or for processing errors if said threshold is exceeded.

Alternatively or additionally, sensor arrangement calibration information 528 can be provided, by means of which properties of the sensor arrangement 113 or sensor apparatus 113 are or can be set. In particular, it is provided that the sensor arrangement calibration information 528 is transmitted or can be transmitted to the sensor arrangement 113 or sensor apparatus 113 by the analysis device 200, and that the sensor arrangement 113 or sensor apparatus carries out or is designed to carry out a measurement taking into account the sensor arrangement calibration information 528.

The proposed analysis system 1 preferably comprises evaluation information 530 which is stored in the database 500 and/or is retrievable or can be retrieved from the database 500. The evaluation information 530 is preferably designed to be able to interpret measurement results 713 that originate from the cartridge 100, in particular from the sensor apparatus 113.

The control information 510 and/or the evaluation information 530 particularly preferably comprises instructions, preferably in the form of an algorithm and/or for controlling a process on or using a processor or controller. The instructions preferably form a module that can be or is implemented by the analysis device 200 and/or the operating instrument 400, as a result of which the behavior of the analysis device 200 and/or the operating instrument 400 can be or is changed.

The instructions are in particular commands, machine code, pre-compiled source code or source code. The instructions preferably form a module-like software component, in particular a plugin. The instructions can be designed to form and/or to replace a module of the operating instrument 400 and/or of the analysis device 200. For this purpose, the control information 510 and/or the evaluation information 530 can comprise a (software) interface for coupling or implementation by the control apparatus 207 and/or an evaluation module 440 of the operating instrument 400.

The control information 510 particularly preferably comprises or forms a module of the control apparatus 207 that can be exchanged, preferably in terms of software. This module preferably contains instructions such as logic commands, loops and the like for controlling the test, in particular in the form of a computer program or computer program product to be executed by the analysis device 200 and/or the control apparatus 207. The control information 510 can be or form, in particular as a plugin, an exchangeable part of the control apparatus 207.

An evaluation module 440 is preferably formed by the operating instrument 400 or the operating instrument 400 comprises the evaluation module 440. By means of the evaluation module 440, measurement results 713 read out from the sensor apparatus 113 are evaluated preferably using the evaluation information 530 and/or the evaluation module 440 is designed for this purpose.

The evaluation information 530 particularly preferably comprises or forms a module of the evaluation apparatus 440 that can be exchanged, preferably in terms of software. This module preferably contains instructions such as logic commands, loops and the like for controlling the evaluation of measurement results 713, in particular in the form of a computer program or computer program product to be executed by the operating instrument 400 and/or the evaluation module 440. The evaluation information 530 can be or form, in particular as a plugin, an exchangeable part of the evaluation module 440.

Alternatively or additionally, the instructions can comprise parameters for configuring the control apparatus 207 and/or the evaluation module 440. These parameters are preferably provided in addition to the instructions, for example for the analysis device 200 in the form of or comprising the calibration information 520. Alternatively, the control information 510 and/or evaluation information 530 can however also merely comprise parameters and/or other information for the control and/or evaluation.

The database 500 preferably comprises a results memory 550 in which results can be stored and/or saved.

Within the meaning of the present invention, the term “database” should preferably be understood in a broad sense and also incorporates multi-part databases in particular. Therefore, in principle, the database 500 can be provided in different physical units or at different locations and/or can be composed of a plurality of subdatabases.

The operating instrument 400 can preferably be separated and/or disconnected from the analysis device 200 with respect to a data connection and/or physically. For this purpose, the analysis device 200 can initially be connected to the operating instrument 400 by the data connection DVA being established.

In order to control the test and/or the analysis device 200, the operating instrument 400 can retrieve control information 510 from the database 500 and transmit said information to the analysis device 200 in unaltered or altered form.

The operating instrument 400 is preferably designed to evaluate measurement results 713 which can preferably be generated by the sensor apparatus 113 of the cartridge 100 while the sample P is being tested. For this purpose, it is provided that measurement results 713, which can originate from a sensor apparatus 113 of the cartridge 100 and/or which can be transmitted from the analysis device 200 to the operating instrument 400, are or can be evaluated in the operating instrument 400. For this purpose, the operating instrument 400 can retrieve or receive the evaluation information 530 from the database 500 and, using this evaluation information 530, evaluate the measurement results 713, in particular in the evaluation module 440 of the operating instrument 400.

The operating instrument 400 preferably comprises a memory 450. The memory 450 can be used to store, at least temporarily, control information 510, calibration information 520 and/or evaluation information 530, or the operating instrument 400 and the memory 450 can be designed for this purpose. Alternatively or additionally, evaluation results 740, that have been or can be generated from the measurement results 713 by means of the operating instrument 400, can be stored in the memory 450.

In one example, the operating instrument 400 comprises an output apparatus 410, preferably an in particular touch-sensitive screen or display 411 and/or a speaker 412. Alternatively or additionally, the operating instrument 400 comprises an input apparatus 420, in particular a camera 421, a touchpad 422, a microphone 423 and/or a keyboard 424.

The operating instrument 400 is preferably designed to display on operating interface or a user interface via the output apparatus 410, in particular the screen or display 411, or to provide in another way operating elements for controlling the test and/or the analysis device 200, and/or to output a status or other information relating to the test. Alternatively or additionally, commands can be received via the input apparatus 420, by means of which the operating instrument 400 starts, configures and/or controls the test of the sample P in a manner corresponding to the commands.

Preferably, the transmission of commands and/or information to the analysis device 200 is triggered via the input apparatus 420 or can be triggered by the input apparatus 420.

In particular, transmission of the control information 510 from the operating instrument 400 to the analysis device 200 can be initiated or controlled via the input apparatus 420. Alternatively or additionally, the analysis device 200 can be controlled in order to receive the cartridge 100 and/or to start the test, preferably using the control information 510 and/or a command received via the input apparatus 420.

The operating instrument 400 is preferably designed to transmit, to the analysis device 200, control information 510 for receiving or ejecting the cartridge 100. In this case, a cartridge 100 can in particular be inserted only when the operating instrument 400 is connected to the analysis device 200, whereupon the operating instrument 400 can verify the cartridge 100 and can eject said cartridge or block a test if an error, such as incompatibility, is detected.

Alternatively or additionally, the operating instrument 400 is designed to transmit control information 510 for starting the test to the analysis device 200. The test is thus preferably started only by a command originating from the operating instrument 400. The analysis device 200 itself preferably does not comprise a user interface for generating a start command or for causing the test to start. This task is preferably reserved for the operating instrument 400.

The cartridge 100 preferably comprises at least one cartridge identifier 100C which corresponds to the cartridge 100 and/or to a batch with which the cartridge 100 is associated.

The cartridge identifier 100C is in particular a piece of information that is specific to the relevant cartridge 100, is in particular unique and/or is designed to uniquely identify the cartridge 100, such as an identification code which is assigned to the relevant cartridge 100 and makes it possible for said cartridge to be identified in a preferably unique manner.

Alternatively or additionally, the cartridge identifier 100C makes it possible to assign the cartridge 100 to a production cycle and/or to a batch of particular cartridges 100. A batch is preferably characterized in that cartridges 100 are produced in the same continuous production cycle and/or are produced having the same components, in particular having the same sensor apparatuses 113 and/or the same reagents and the like. There is preferably a plurality of batches which can differ from one another with regard to production periods, batches of starting materials used and the like, for example.

The cartridge identifier 100C can be stored and/or saved in a memory means 100D of the cartridge 100. The memory means 100D can be a barcode 124, an NFC tag and/or a memory which is provided in the sensor apparatus 113, is connected to the sensor apparatus 113 or is assigned to the sensor apparatus 113, or another apparatus for storing code or the like.

The cartridge identifiers 100C are preferably assigned to the respective cartridges 100. In particular, the cartridge identifier 100C is formed by the cartridge 100, connected thereto and/or arranged thereon.

The analysis system 1 can comprise a plurality of cartridges 100 which can each preferably be distinguished from one another by means of at least one cartridge identifier 100C and/or which are assigned to a batch.

Alternatively or additionally, the same cartridge 100 can comprise at least two cartridge identifiers 100C that each correspond to the cartridge 100. The cartridge identifiers 100C can preferably be read out by different read-out methods, in particular optically, by radio, by a wired connection or the like.

The respective cartridges 100 can comprise two different memory means 100D having the same or corresponding cartridge identifiers 100C. The memory means 100D are preferably independent of one another and/or separated from one another physically. The memory means 100D can preferably be read out in different ways, in particular electronically and/or by an electronic connection on the one hand, and wirelessly, in particular optically and/or by radio on the other hand.

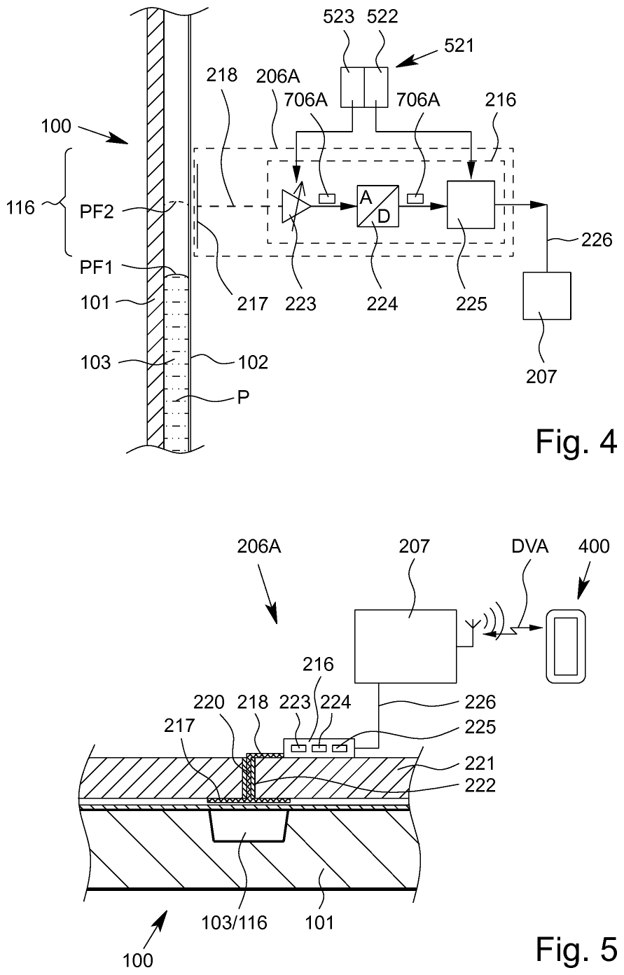

FIG. 4 is a schematic view of the fluid sensor 206A and a detail of the fluid system 103 of the cartridge 100 in the region of a sensor portion 116.

The sensor portion 116 can be formed by a cross-sectional widening of a channel 114 of the fluid system 103, as shown in FIG. 2 by way of example. In particular, it is provided that the inside diameter is larger than that of adjacent channels in the direction of the main plane of extension of the main body 101 of the cartridge 100 and/or transversely to a flow direction that is assigned to the sensor portion 116 and connects an inlet and an outlet of the sensor portion 116. This makes it possible for a liquid front to be formed that is wider and thus has a higher degree of detectability in comparison with adjacent channels 114.

The sensor portion 116 preferably comprises (exactly) one inlet and (exactly) one outlet that have a preferably continuous and/or steady cross-sectional widening and/or cross-sectional tapering in order to prevent flow separations and/or turbulences. In principle, however, other constructions of the sensor portion 116 are also conceivable, although the described construction is preferred owing to a particularly high degree of detectability of content changes, in particular liquid fronts PF1, PF2.

In FIG. 4, the fluid system 103 comprises a gas, in particular (conditioned) air or the like, in the sensor portion 116. Furthermore, the sample P or another liquid, which can be conveyed within the fluid system 103 of the cartridge 100, is located at other points in the fluid system 103. For details in this regard, reference is made to the description of FIGS. 1 and 2.

The sample P or another liquid that is located and/or can be conveyed in the fluid system 103 preferably comprises or forms a boundary layer or front or liquid front PF1, preferably at a layer forming a boundary with the atmosphere in the fluid system 103 or with another fluid. The liquid front PF1 preferably extends at least substantially transversely to the flow direction and/or longitudinal extension of the sensor portion 116.

The liquid front PF1 can be moved within the fluid system 103 and/or within the sensor portion 116 by means of the sample P or the fluid that forms the liquid front PF1 being conveyed. In the example shown according to FIG. 4, the front PF1 migrates into the sensor portion 116 by means of the sample P or another fluid being conveyed, and in the process displaces the gas or another fluid that was previously located in the sensor portion 116, as shown by the liquid front PF2 marked out with a dashed line.

The content of the sensor portion 116 is changed by the displacement. This content change in the sensor portion 116 can then be detected by the fluid sensor 206A.

Owing to the fact that the liquid front PF1, PF2 is continuously moving in the sensor portion 116 and through the sensor portion 116, a continual content change can preferably be determined and evaluated using the fluid sensor 206A.

The fluid sensor 206A preferably operates electrically. For this purpose, the fluid sensor 206A preferably comprises a sensor electrode 217 which is preferably arranged in the analysis device 200, when the cartridge 100 is loaded, adjacently to the sensor portion 116 such that the electrical properties of the sensor electrode 217 are or can be influenced by the content of the sensor portion 116. In particular, the electrical properties are or can be changed by displacing the atmosphere and replacing it with the sample P or a liquid such that it is possible to identify that the liquid or sample P has reached the sensor portion 116.

Particularly preferably, the fluid sensor 206A operates capacitively. For this purpose, the sensor electrode 217 has a preferably plate-like or plate-shaped construction of which a flat side faces the sensor portion 116. On account of the sensor electrode 217 being arranged adjacently to the sensor portion 116, the ability of said sensor electrode to absorb and store charges is dependent on the permittivity, in particular the relative permittivity, of the cartridge 100 and thus also on the content of the sensor portion 116. The fluid sensor 206A therefore detects the content change in the sensor portion 116 preferably indirectly on the basis of the change in the capacitance of the sensor electrode 217 resulting from a change in the permittivity of the content of the sensor portion 116.

The sensor electrode 217 preferably comprises just one or exactly one pole, i.e. in particular exactly one sensor plate. This is advantageous in that a counter electrode does not need to be used. This simplifies the construction and arrangement of the fluid sensor 206A in complex systems in particular.

The sensor electrode 217 is preferably designed to measure an electrical variable, in particular capacitance, which is dependent on an electrical property of the content of the sensor portion 116, said electrical property preferably being the permittivity, in particular the relative permittivity, of the content, which influences the electrical properties of the sensor electrode 217 and thus influences, preferably changes, the measurement result 706A.

Alternatively or additionally, it is however also possible for the fluid sensor 206A to measure electrical conductivity of the content of the sensor portion 116. The change in the electrical conductivity of the content of the sensor portion 116 can be determined for example by electric and/or magnetic fields being coupled into the content of the sensor portion 116. Depending on the electrical conductivity, electrical currents are induced that are converted into heat in a manner dependent on the electrical conductivity, and this can be measured by the fluid sensor 206A, in particular as a loss.

In principle, the fluid sensor 206A can, alternatively or additionally, also be operated inductively, the content change in the sensor portion 116 preferably being determined by so-called eddy current losses. Here too, the change in the electrical properties of the sensor electrode 217 is preferably determined depending on the content of the sensor portion 116, in particular depending on the electrical conductivity. This process preferably utilizes the fact that different conductivities of the content of the sensor portion 116 lead to different degrees of ability for inducing eddy currents and/or to different eddy current losses.

The term “eddy current loss” preferably denotes the effect whereby, when an eddy current is induced, owing to the finite electrical conductivity or the electrical resistance of the content of the sensor portion 116, energy is converted into heat energy and can be measured by the fluid sensor 206A as electrical energy loss.

The fluid sensor 204A preferably has evaluation electronics 216 for detecting the content change in the sensor portion 116 of the cartridge 100 and/or for processing and/or evaluating the signal from the sensor electrode 217 or measurement result 706A.

The sensor electrode 217 is preferably connected to the evaluation electronics 216 by means of a sensor line 218.

The evaluation electronics 216 preferably comprises a measuring amplifier 223. The measuring amplifier 223 can be designed to measure the electrical property that can be influenced by the content of the sensor portion 116, in particular the capacitance of the sensor electrode 217. Particularly preferably, the measuring amplifier 223 is designed to amplify a detected change in the electrical property of the sensor electrode 217 in order to be able to subsequently evaluate or interpret said change with a higher degree of accuracy.

The evaluation electronics 216, in particular the measuring amplifier 223, is preferably designed to carry out what is known as zero-point adjustment or offset adjustment. In this case, once the cartridge 100 has been loaded into the analysis system 200, the electrical property, in particular capacitance, of the arrangement comprising or formed by the sensor electrode 217 and the sensor line 218 is determined and/or compensated for in an initial state. In particular, this electrical property or capacitance is cancelled out in the evaluation electronics 216 by countermeasures and/or used as the zero point, in order for it to be possible to determine and/or output, on this basis, the slightest of changes in the electrical properties of the sensor electrode 217.

The evaluation electronics 216 preferably comprises an analogue-to-digital converter 224, also referred to as an A/D converter 224. The A/D converter 224 converts the measuring signal, which is preferably initially present in the analogue form and which corresponds to the change in the electrical property of the sensor electrode 217 and indirectly to the content change in the sensor portion 116, into a digital signal. However, in principle, the A/D converter 224 can also be omitted and an evaluation can be carried out using analogue signals.

The analysis device 200 preferably comprises a detection apparatus 225 for detecting the change in the electrical property of the sensor electrode 217. In the example shown, the detection apparatus 225 is formed by the evaluation electronics 216 or the evaluation 216 comprises the detection apparatus 225. In principle, the detection apparatus 225 can however also be formed so as to be separated from the evaluation electronics 216 and/or so as to be part of the control apparatus 207.

The detection apparatus 225 is preferably designed to evaluate the measurement result 706A in order to detect a content change in the sensor portion 116. Particularly preferably, the detection apparatus 225 analyses a course or profile of or curve for the measurement result 706A and/or compares the measurement result 706A with a reference value.

The evaluation electronics 216 forwards the result of the evaluation by the detection apparatus 225 to the control apparatus 207 preferably via a control interface 226. The control apparatus 207 can control the test, in particular activate actuators, in a manner dependent on the content change in the sensor portion 116 being detected, in order to convey the sample P, to temperature-control the sample, to conduct the sample within the cartridge 100 in a certain way and/or to analyze the sample.

It is particularly preferably provided that the detection apparatus 225 transmits a signal to the control apparatus 207 via the control interface 226 as soon as a content change, in particular a content change in the sensor portion 116 that is expected or is in line with expectations in the test sequence, has been detected.

The control information 510 and/or execution information 511 can comprise condition information 511C which is designed to control the test in a particular manner dependent on the event of the signal being transmitted and/or the content change being identified.

In one aspect of the present invention, which can also be implemented independently, it is provided that the detection apparatus 225 is configured and/or that the configuration is changed depending on a measurement result expected in the test sequence. In particular, the detection apparatus 225 compares the measurement result with a reference value and/or threshold value 522 which is or can be set and/or is or can be specified in a manner dependent on the cartridge 100, the test to be carried out and/or the phase of the test. This makes it possible to fix, define or adapt the reference value and/or threshold value 522 for a content change that is expected or is to be expected.

In particular, the analysis system 1 comprises a plurality of different cartridges 100, supports a plurality of different cartridges 100 and/or supports a plurality of different tests. In the same or different tests, test phases and/or test steps, it can be provided that different substances, preferably liquids, in particular samples P, wash buffers, reagents, etc., are conveyed within the fluid system 103, which substances influence the electrical properties of the sensor electrode 217 in different ways, in particular sometimes to a greater extent and sometimes to a lesser extent, when they reach the sensor portion 116. This can be taken into account by the detection apparatus 225, particularly preferably by different reference values and/or threshold values 522 being used or by the reference value and/or threshold value 522 being changed or adapted.

It is also possible for different content changes to be provided during a test using the same cartridge 100, for example when different substances, such as the sample P, wash buffer, reagents, gases and the like, arrive at the sensor portion 116 alternately and/or in succession.

During the test, at least two, preferably at least three or more, different measurement results 706A are therefore expected. Consequently, the reference value and/or threshold value 522 can be fixed, defined and/or adapted preferably such that each of the expected content changes in the sensor portion 116 can be detected reliably and/or with an improved degree of accuracy. This does not mean that, in this case, the fluid sensor 206A determines the content of the sensor portion 116, even though this would also be possible in principle, but rather it means that the sensitivity, thresholds and the like for the sensor electronics 216 can be adapted in order to improve reliable detection of a content change.

As an alternative or in addition to the reference value and/or threshold value 522, the gain 523 of the measuring amplifier 223 can be set, specified and/or varied, it being possible for the criteria and procedures therefor to correspond to the specification and/or adaptation of the reference value and/or threshold value 522, and so reference is made to corresponding explanations.

The fluid sensor calibration information 521 preferably comprises the reference value and/or threshold value 522 and/or the gain 523.

As already explained in conjunction with FIG. 3, the fluid sensor calibration information 521, in particular the reference value and/or threshold value 522 and/or the gain 523, can be retrieved and/or transmitted, in particular sent, to the analysis device 200. For this purpose, the fluid sensor calibration information 521 can be retrieved from the database 500 and/or transmitted to the analysis device 200 by means of the operating instrument 400.

In this respect, it is particularly preferable for in particular the operating instrument 400 to determine or establish the cartridge identifier 100C of the cartridge 100, in particular to read out said identifier 100C, for example from the barcode 124 of the cartridge 100 using the camera 421.

Using the cartridge identifier 100C, the operating instrument 400 and/or the database 500 can identify the fluid sensor calibration information 521 corresponding to the cartridge 100 and/or a test that can be carried out using the cartridge 100, and transmit said information to the analysis device 200. The fluid sensor calibration information 521 can form part of the control information 510 and/or the calibration information 520.

As described in conjunction with FIG. 3, the fluid sensor calibration information 521 can therefore be retrieved or transmitted as part of the control information 510. However, in principle, it is conceivable for the fluid sensor calibration information 521 to also be handled separately from the control information 510.

In particular, the control information 510 contains calibration information 520, i.e. in particular the fluid sensor calibration information 521, as part of execution information 511, and therefore, when the test is being carried out, the reference value and/or threshold value 522 and/or the gain 523 can be adapted on the basis of the control information 510.

In general, it is therefore preferable for the sensitivity of the evaluation electronics 216 to be specified and/or changed using the calibration information 520 depending on the selection of one of a plurality of possible cartridges 100 and/or depending on the selection of a specific test and/or the phase of a test sequence, the calibration information 520 in particular being established, determined, retrieved and/or used on the basis of the cartridge identifier 100C.

FIG. 5 is a schematic section through the fluid sensor 206A and a detail of the cartridge 100 that comprises the main body 101 which forms the fluid system 103, in this case in the region of the sensor portion 116.

The sensor electrode 217 is arranged adjacently to the sensor portion 116 such that the content or a content change in the sensor portion 116 can be detected by means of the fluid sensor 206A.

The sensor electrode 217 is connected to the evaluation electronics 216 by means of the sensor line 218.

Preferably, only one sensor electrode 217 is provided, i.e. what is known as a single-ended arrangement that does not comprise a counter electrode. In principle, other solutions are also possible.

The sensor line 218 preferably comprises a via 220 in a printed circuit board 221 of the analysis device 200. The evaluation electronics 216 is preferably arranged on a side of the printed circuit board 221 that is remote from the sensor electrode 217. This provides, in a simple manner, for a construction or design that is at least substantially flat or planar on the side of the printed circuit board 221 that faces the cartridge 100, the construction being advantageous for positioning the printed circuit board 221 flat against the cartridge 100 and/or for effective coupling of the sensor electrode 217 to the cartridge 100.

The evaluation electronics 216 is preferably provided in a housing, in particular in a dual in-line package housing, an SMD housing and/or a BGA housing. In this case, the evaluation electronics 216, including the housing, is arranged on the side of the printed circuit board 221 that is remote from the sensor electrode 217. This is advantageous in terms of construction in that the evaluation electronics 216 does not increase the distance between the sensor electrode 217 and the cartridge 100.

The sensor line 218 is preferably surrounded by a dielectric 222 in the region of the via 220. The dielectric 222 can be arranged or provided next to, preferably around, the sensor line 218, in particular in the manner of a sleeve. The dielectric 222 can be separated from the printed circuit board 221 and/or can be formed through the printed circuit board 221.

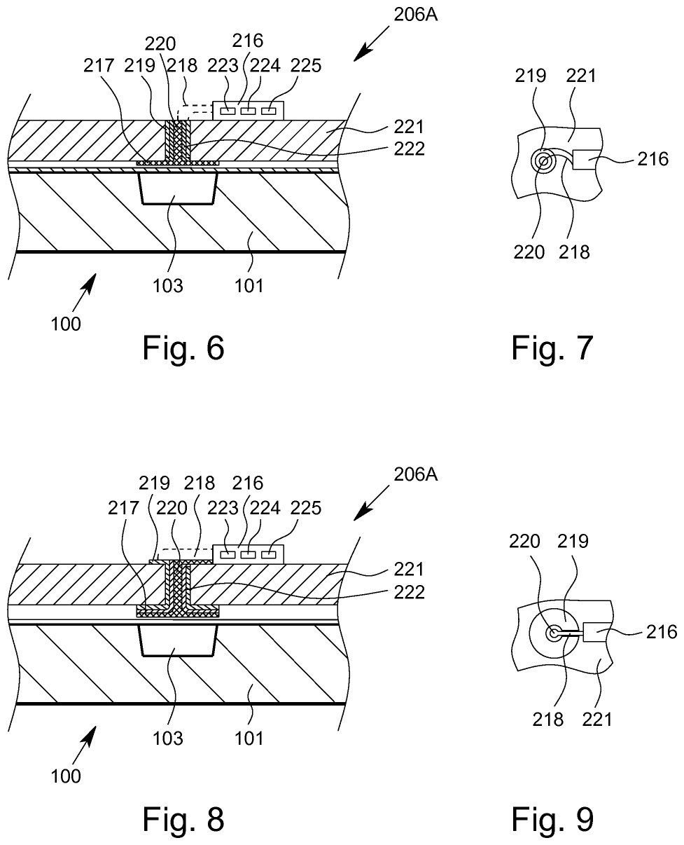

FIG. 6 shows an alternative, preferred embodiment of the proposed fluid sensor 206A, again in a schematic section, a shield electrode 219 being assigned to the sensor line 218. In particular, the shield electrode 219 extends adjacently to the sensor line 218. This makes it possible to prevent interfering signals from being coupled into the sensor line 218.

Alternatively or additionally, the shield electrode 219 reduces the influence or influenceability of the capacitance and/or capacitor formed by the sensor electrode 217 and the sensor line 218 in the region of the sensor line 218. This can be achieved by the shield electrode 219 forming, together with the sensor line 218, an at least substantially invariable and/or constant electrical capacitance. Influences on the evaluation of the measurement results 706A that could potentially lead to an incorrect interpretation, arising as a result of the capacitance of the arrangement, consisting of the sensor electrode 217 and the sensor line 218, when there is a change in the properties of the surroundings at a greater distance than the distance between the sensor line 218 and the shield electrode 219, are thus prevented.

The shield electrode 219 is preferably electrically connected to the evaluation electronics 216 and/or to earth. This can be achieved for example by lines provided on the printed circuit board 221. Alternatively or additionally, wires, for example bonding wires, can be used, as is shown by way of example in the very schematic detail from FIG. 7.

The shield electrode 219 can surround the sensor line 218 in the region of the via 220, in particular in the manner of a sleeve. In order to prevent direct electrical, i.e. galvanic, contact between the shield electrode 219 and the sensor line 218, the dielectric 222 is preferably provided therebetween. The shield electrode 219 is therefore preferably always isolated from the sensor line 218.

The sensor electrode 217 according to FIG. 6 is arranged directly on the printed circuit board 221. In particular, the sensor electrode 217 is formed and/or structured by structuring a metal lamination of the printed circuit board 221, in particular a copper lamination. This makes it possible for the sensor electrode 217 to have a planar construction.

FIG. 8 shows another variant of the fluid sensor 206A, the shield electrode 219 being guided in parallel with the sensor electrode 217 on the side facing the cartridge 100. In this case, the sensor electrode 217 preferably has a multilayered construction, the sensor electrode 217 in particular being provided on a side of the dielectric 222 that faces the cartridge 100 and the shield electrode 219 in particular being provided on a side of the dielectric 222 that is remote from the cartridge 100.

As shown in the very schematic top view according to FIG. 9, the shield electrode 219 can cover the sensor electrode 217 on the side that is remote from the cartridge 100, preferably at least substantially completely and/or in a projecting manner.

Furthermore, the shield electrode 219 is preferably guided on the printed circuit board 221, in particular at a short distance from and/or at least substantially in parallel with the sensor line 218. The sensor line 218 is preferably formed as a conducting track on the printed circuit board 221. The shield electrode 219 is also preferably formed on the printed circuit board 221 at least in part. This can be achieved by the metal surface or lamination of the printed circuit board 221 being structured in a corresponding manner.

The sensor line 218 and the shield electrode 219 extend on the printed circuit board 221, in particular on the flat side thereof that is remote from the cartridge 100, preferably in the same plane and/or next to one another.

In order to prevent galvanic contact between the sensor line 218 and the shield electrode 219, the shield electrode 219 can surround the sensor line 218 in the region of the via 220 and be guided, preferably in an uninterrupted manner, between the via 220 and a connection point for the sensor electronics 216 so as to be adjacent and/or parallel to the sensor line 218.

In the region of the via 220, the shield electrode 219 is preferably arranged coaxially with the sensor line 218. However, it is not absolutely necessary for the shield electrode 219 to completely surround the sensor line 218.

In general, the analysis device 200, the cartridge 100 or in particular the sensor apparatus 113 may measure, detect or identify the one or more analytes A by means of specific bonding, in particular by means of capture molecules and/or of means of electrochemical detection such as redox cycling, or the like, preferably performed on the cartridge 100 and/or in the sensor apparatus 113. Preferably, the capture molecules are arranged or immobilized on a sensor array or on sensor fields or electrodes of the sensor apparatus 113. In particular, an immuno-assay or a protein assay for detecting or identifying a protein and/or a nucleic-assay for detecting or identifying a nucleic-acid sequence can be or is realized.

Alternatively or additionally, measurements without specific bonding and/or without electrochemical detection can be used or performed, preferably in or by the analysis device 200 and/or cartridge 100. Such measurements can include an optical measurement, impedance measurement, capacitance measurement, spectrometric measurement, mass spectrometric measurement, or the like. For this purpose, the analysis device 200 or cartridge 100 may comprise an optical spectrometer and/or allow optical measurements of the treated or untreated sample P. Thus, it is possible to measure, detect or identify other or further analytes A, compounds, material characteristics, or the like of the sample P, e.g. within the cartridge 100 or any other sample carrier. These alternative or additional measurements can be used or processed and/or evaluated in a similar manner as described or differently.

Individual aspects and features of the present invention and individual method steps and/or method variants may be implemented independently from one another, but also in any desired combination and/or order.

In particular, the present invention relates also to any one of the following aspects which can be realized independently or in any combination, also in combination with any aspects described above:

1. Method for testing an in particular biological sample P by means of an analysis system 1, the analysis system 1 comprising a cartridge 100 for receiving the sample P, the cartridge 100 comprising a fluid system 103 that has a sensor portion 116 through which fluid can flow, the analysis system 1 comprising an analysis device 200 for receiving the cartridge 100 and subsequently carrying out the test using the received cartridge 100, the analysis device 200 comprising at least one fluid sensor 206A that has evaluation electronics 216 for detecting a content change in the sensor portion 116, characterized in that the sensitivity of the evaluation electronics 216 is specified and/or changed depending on a phase of the test sequence and/or depending on a cartridge identifier 100C of the cartridge 100, and/or in that the fluid sensor 206A comprises a sensor electrode 217 that is intended for measuring electrical capacitance and is operated single ended and/or electrically connected to the evaluation electronics 216 by a shielded sensor line 218.
2. Method according to the preceding aspect, characterized in that the fluid sensor 206A measures, as a measurement result 706A, an electrical variable, in particular capacitance, which is dependent on a property, in particular permittivity and/or electrical conductivity, of the content of the sensor portion 116.
3. Method according to aspect 2, characterized in that if the measurement result 706A changes, it is concluded that there has been a content change in the sensor portion 116, the measurement result 706A preferably being compared with a reference value and/or threshold value 522 and the content change being detected if the reference value and/or threshold value 522 is exceeded.
4. Method according to any one of the preceding aspects, characterized in that the sensitivity of the evaluation electronics 216 is defined by a reference value and/or threshold value 522 for comparison with a measurement result 706A originating from the fluid sensor 206A and/or by a gain 523 of a measuring amplifier (223) connected to the sensor electrode 217.
5. Method according to aspect 4, characterized in that the reference value 522 and/or the gain 523 is specified and/or changed depending on the phase of the test sequence and/or depending on the cartridge identifier 100C of the cartridge 100.
6. Method according to any one of the preceding aspects, characterized in that calibration information 520 corresponding to the cartridge 100 is stored, preferably comprising the reference value and/or threshold value 522 and/or the gain 523, the calibration information 520 corresponding to the cartridge 100 is retrieved and/or determined by means of the cartridge identifier 100C, and the sensitivity of the evaluation electronics 216 preferably being set using the calibration information 520.
7. Method according to any one of the preceding aspects, characterized in that a liquid front PF1, PF2 moving within the sensor portion 116 is detected.
8. Method according to any one of the preceding aspects, characterized in that the test on the sample P, in particular conveyance of the sample P and/or actuation of valves 115, is controlled in a manner dependent on the detection of a content change in the sensor portion 116, in particular on the detection of the liquid front PF1, PF2 moving within the sensor portion 116.
9. Computer program product comprising program code means which, when executed, cause the method steps of the method according to any one of the preceding aspects to be carried out.
10. Analysis system 1 for testing an in particular biological sample P, said analysis system preferably being designed to carry out the method according to any one of the preceding aspects,
the analysis system 1 comprising a cartridge 100 for receiving the sample P, the cartridge 100 comprising a fluid system 103 that has a sensor portion 116 through which fluid can flow, the analysis system 1 comprising an analysis device 200 for receiving the cartridge 100 and subsequently carrying out the test using the received cartridge 100, the analysis device 200 comprising at least one fluid sensor 206A that has evaluation electronics 216 for detecting a content change in the sensor portion 116, characterized in that the analysis system 1 is designed to specify and/or change the sensitivity of the evaluation electronics 216 depending on a phase of the test sequence and/or depending on a cartridge identifier 100C of the cartridge 100, and/or in that the fluid sensor 206A comprises a sensor electrode 217 that is intended for measuring electrical capacitance and is single ended and/or electrically connected to the evaluation electronics 216 by a shielded sensor line 218.
11. Analysis system according to aspect 10, characterized in that the evaluation electronics 216 is designed to measure the electrical capacitance of the sensor electrode 217, preferably without using a reference electrode or counter electrode.
12. Analysis system according to aspect 10 or 11, characterized in that a shield electrode 219 is provided adjacently to the sensor line 218.
13. Analysis system according to any one of aspects 10 to 12, characterized in that the sensor line 218 connects the sensor electrode 217 to the evaluation electronics 216 by means of a via 220 in a printed circuit board 221.
14. Analysis system according to aspect 13, characterized in that the sensor line 218 is shielded in the region of the via 220, preferably by the shield electrode 219, the shield electrode 219 in particular being arranged coaxially with the via 220.
15. Analysis system according to any one of aspects 10 to 14, characterized in that an at least substantially constant electrical capacitance is produced between the sensor line 218 and the shield electrode 219, the evaluation electronics 216 preferably being designed to measure and/or compensate for this constant capacitance and/or to ignore said capacitance in the evaluation.

1-48. (canceled)

49. A method for testing a sample by means of an analysis system, comprising:
receiving the sample in a cartridge of the analysis system, the cartridge comprising a fluid system that has a sensor portion through which fluid can flow; and
receiving the cartridge by an analysis device of the analysis system, and subsequently carrying out a test using the received cartridge, the analysis device comprising at least one fluid sensor that has evaluation electronics for detecting a content change in the sensor portion,
wherein the sensitivity of the evaluation electronics is at least one of specified or changed depending on one or more of a phase of a test sequence and a cartridge identifier of the cartridge.

50. The method according to claim 49, wherein the fluid sensor comprises a sensor electrode that is configured for measuring electrical capacitance electrically connected to the evaluation electronics by a shielded sensor line.

51. The method according to claim 49, wherein the fluid sensor measures, as a measurement result, one or more of an electrical variable or capacitance, the electrical variable or capacitance being dependent on one or more of a property of the content of the sensor portion, permittivity of the content of the sensor portion, electrical conductivity of the content of the sensor portion, and a liquid front moving within the sensor portion.

52. The method according to claim 51, wherein if the measurement result changes, one or more of the following is performed: it is concluded that there has been a content change in the sensor portion; the measurement results are compared with a reference value or threshold value; and the content change is detected if at least one of a reference value or threshold value is exceeded.

53. The method according to claim 49, wherein the sensitivity of the evaluation electronics is defined by one or more of a reference value for comparison with a measurement result originating from the fluid sensor, a threshold value for comparison with a measurement result originating from the fluid sensor, and a gain of a measuring amplifier connected to the sensor electrode.

54. The method according to claim 53, wherein the one or more of the reference value, the threshold value, and the gain is specified or changed depending on the phase of the test sequence.

55. The method according to claim 53, wherein the one or more of the reference value, the threshold value, and the gain is specified or changed depending on the cartridge identifier of the cartridge.

56. The method according to claim 49, wherein calibration information corresponding to the cartridge is stored, the calibration information corresponding to the cartridge is at least one of retrieved or determined by means of the cartridge identifier, and the sensitivity of the evaluation electronics is set using the calibration information.

57. The method according to claim 56, wherein the calibration information comprises one or more of the reference value, the threshold value and the gain, and the sensitivity of the evaluation electronics is set using the one or more of the reference value, the threshold value and the gain.

58. The method according to claim 49, wherein one or more of the test on the sample, conveyance of the sample, and actuation of valves is controlled in a manner dependent on the detection of a content change or a liquid front moving in the sensor portion.

59. The method according to claim 49, wherein an at least substantially constant electrical capacitance is produced between the sensor line and a shield electrode, wherein the evaluation electronics one or more of measures or compensates for this constant capacitance, and ignores said constant capacitance in evaluation.

60. A computer program product comprising program code which, when executed, causes the method steps of the method according to claim 49 to be carried out.

61. An analysis system for testing a sample, comprising:
a cartridge for receiving the sample, the cartridge comprising a fluid system that has a sensor portion through which fluid can flow; and
an analysis device for receiving the cartridge and subsequently carrying out a test using the received cartridge, the analysis device comprising at least one fluid sensor that has evaluation electronics for detecting a content change in the sensor portion,
wherein the analysis system is configured to at least one of specify or change the sensitivity of the evaluation electronics depending on one or more of a phase of the test sequence and a cartridge identifier of the cartridge.

62. The analysis system according to claim 61, wherein a shield electrode is provided adjacently to the sensor line, wherein an at least substantially constant electrical capacitance is produced between the sensor line and the shield electrode, wherein the evaluation electronics is designed to one or more of measure the constant capacitance, compensate for the constant capacitance, and to ignore the constant capacitance in the evaluation.

63. A method for testing a sample by means of an analysis system, comprising:
receiving the sample in a cartridge of the analysis system, the cartridge comprising a fluid system that has a sensor portion through which fluid can flow; and
receiving the cartridge at an analysis device of the analysis system, and subsequently carrying out a test using the received cartridge, the analysis device comprising at least one fluid sensor that has evaluation electronics for detecting a content change in the sensor portion,
wherein the fluid sensor comprises a sensor electrode that is configured for measuring electrical capacitance and is at least one of operated single ended or electrically connected to the evaluation electronics by a shielded sensor line.

64. The method according to claim 63, wherein the fluid sensor measures, as a measurement result, one or more of an electrical variable and a capacitance, the one or more of electrical variable and capacitance being dependent on one or more of a property of the content of the sensor portion, permittivity of the content of the sensor portion, electrical conductivity of the content of the sensor portion, and a liquid front moving within the sensor portion.

65. The method according to claim 63, wherein one or more of the test on the sample, the conveyance of the sample, and actuation of valves is controlled in a manner dependent on the detection of a content change in the sensor portion.

66. The method according to claim 63, wherein one or more of the test on the sample, the conveyance of the sample, and actuation of valves is controlled in a manner dependent on the detection of the liquid front moving within the sensor portion.

67. An analysis system for testing a sample comprising:
a cartridge for receiving the sample, the cartridge comprising a fluid system that has a sensor portion through which fluid can flow; and
an analysis device for receiving the cartridge and subsequently carrying out a test using the received cartridge, the analysis device comprising at least one fluid sensor that has evaluation electronics for detecting a content change in the sensor portion,
wherein the fluid sensor comprises a sensor electrode that is configured for measuring electrical capacitance and is at least one of single ended or electrically connected to the evaluation electronics by a shielded sensor line.

68. The analysis system according to claim 67, wherein the evaluation electronics is configured to measure the electrical capacitance of the sensor electrode.

69. The analysis system according to claim 67, wherein the sensor line connects the sensor electrode to the evaluation electronics by means of a via in a printed circuit board.

70. The analysis system according to claim 67, wherein an at least substantially constant electrical capacitance is produced between the sensor line and the shield electrode, wherein the evaluation electronics is designed to one or more of measure or compensate for the constant capacitance, and to ignore the constant capacitance in the evaluation.
